(19) United States
(12) Patent Application Publication
(10) Pub. No.: US 2024/0325877 A1
(43) Pub. Date: Oct. 3, 2024

(54) GAME PROGRAM, INFORMATION PROCESSING DEVICE, INFORMATION PROCESSING METHOD, AND INFORMATION PROCESSING SYSTEM

(71) Applicant: The Pokémon Company, Tokyo (JP)

(72) Inventors: Kaname KOSUGI, Tokyo (JP); Marie SHUTO, Tokyo (JP); Yuki TERADA, Tokyo (JP); Koya NAKAHATA, Tokyo (JP); Takumi TSUKADA, Tokyo (JP); Keisuke MIYAGAWA, Tokyo (JP)

(73) Assignee: The Pokémon Company, Tokyo (JP)

(21) Appl. No.: 18/741,850

(22) Filed: Jun. 13, 2024

Related U.S. Application Data
(63) Continuation of application No. PCT/JP2022/045005, filed on Dec. 7, 2022.

(30) Foreign Application Priority Data
Dec. 20, 2021 (JP) 2021-206527

(51) Int. Cl. A63F 13/212; A61B 5/00; A63F 13/79

(52) U.S. Cl. CPC A63F 13/212; A61B 5/4809; A63F 13/79

(57) ABSTRACT

An information processing device for acquiring a sleep state of a user as sleep information is caused to execute acquiring the sleep information of the user a plurality of times; acquiring play information of the user in a game; associating the sleep information with the play information; and determining a plurality of play results on the basis of each combination of the associated play information and sleep information.

### CROSS-REFERENCE TO RELATED APPLICATIONS

This application is a continuation of International Application No. PCT/JP2022/045005, filed Dec. 7, 2022, which claims priority to JP 2021-206527, filed Dec. 20, 2021, the entire contents of each are incorporated herein by reference.

### BACKGROUND

### Technical Field

The present disclosure relates to a game program, an information processing device, an information processing method, and an information processing system.

### Description of Related Art

In recent years, activity measurement devices that measure actions and an amount of activity of a person using a motion detection sensor or the like that detects motions of a body have become widely used. An activity measurement device is designed to improve health awareness of a user by presenting the user with an amount of activity or the like and is desirably used on a continuous basis.

PTL 1 discloses a game system that encourages a user to have appropriate sleep habits. PTL 2 discloses a technique of giving a user a motivation to continuously measure information related to health and check analysis results.

### CITATION LIST

### Patent Literature

[PTL 1] Japanese Patent Application Laid-open No. 2021-48911
[PTL 2] Japanese Patent Application Laid-open No. 2020-72800

### SUMMARY

### Technical Problems

However, although the inventions described in PTL 1 and PTL 2 reflect parameters based on automatically acquired biological information in games, it is not possible to reflect biological information acquired by a plurality of terminals in the games in a case where a user acquires the biological information using the plurality of terminals.

Thus, the present disclosure was made in order to solve the above problems, and an object thereof is to provide a technique of encouraging a user to make an effort to have good sleep habits.

### Solutions to Problems

A game program which a computer that includes a processor and a storage unit and is capable of acquiring a sleep state of a user as sleep information is caused to execute, the game program causing the processor to execute: a sleep acquisition step of acquiring the sleep information of the user corresponding to a plurality of times; a play acquisition step of acquiring play information of the user in a game; an association step of associating the sleep information corresponding to the plurality of times acquired in the sleep acquisition step with the play information acquired in the play acquisition step; and a determination step of determining a plurality of play results on the basis of a combination of the play information and the sleep information corresponding to the plurality of times associated in the association step.

### Advantages

According to the present disclosure, it is possible to reflect sleep information corresponding to one time or multiple times measured by a measurement terminal in a game. It is possible to continuously encourage a user who uses the measurement terminal to make an effort to have good sleep habits.

### BRIEF DESCRIPTION OF DRAWINGS

FIG. 1 is a diagram illustrating an overall configuration of an information processing system.

FIG. 2 is a block diagram illustrating a functional configuration of a server.

FIG. 3 is a block diagram illustrating a functional configuration of a user terminal.

FIG. 4 is a block diagram illustrating a functional configuration of a measurement terminal.

FIG. 5 is a diagram illustrating a data structure of a user table.

FIG. 6 is a diagram illustrating a data structure of a deck table.

FIG. 7 is a diagram illustrating a data structure of a character master.

FIG. 8 is a diagram illustrating a data structure of an item master.

FIG. 9 is a diagram illustrating a data structure of a material master.

FIG. 10 is a diagram illustrating a data structure of a benefit master.

FIG. 11 is a diagram illustrating a data structure of a sleep table.

FIG. 12 is a flowchart illustrating operations in benefit provision processing.

FIG. 13 is a flowchart illustrating operations in sleep synchronization processing.

FIG. 14 is an example of a screen illustrating the operations in the benefit provision processing.

FIG. 15 is an example of a screen presenting a play result in a first presentation mode in the sleep synchronization processing.

FIG. 16 is an example of a screen presenting a play result in a second presentation mode in the sleep synchronization processing.

FIG. 17 is a block diagram illustrating a hardware configuration of a computer.

### DETAILED DESCRIPTION

Hereinafter, embodiments of the present disclosure will be described with reference to the drawings. In all of the diagrams illustrating the embodiments, common components will be denoted by same reference signs and repetitive descriptions will be omitted. It is to be understood that the following embodiments do not unduly restrict the content of the present disclosure as set forth in the scope of claims. In addition, not all components described in the embodiment are essential components of the present disclosure. It is also to be understood that the respective drawings are schematic views and are not necessarily strict illustrations.

### <Overview of Information Processing System 1>

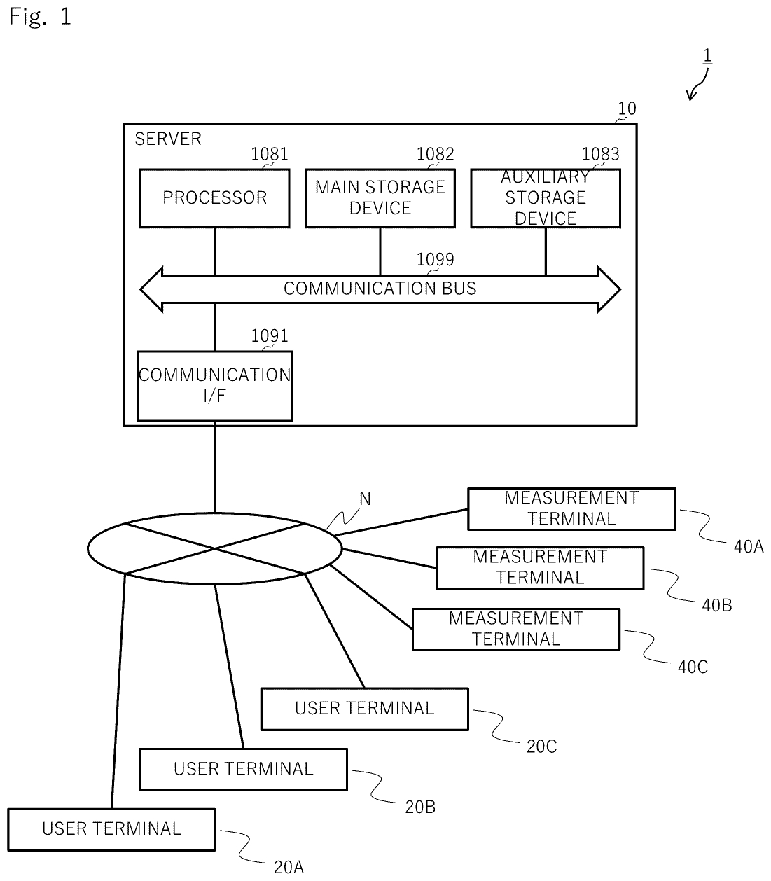

FIG. 1 is a diagram illustrating an overall configuration of an information processing system 1. The information processing system 1 according to the present disclosure is an information processing system that provides a game service that advances on the basis of game operations performed by a user when the user is awake (during daytime, for example) and information related to a sleep state when the user is sleeping (at nighttime, for example). The information processing system 1 is an information processing system for maintaining motivation of the user to have good sleep habits in a process of enjoying the game service.

As the game service provided by the information processing system 1, a growing game will be described as an example in the present disclosure. Note that the present disclosure can be applied to an arbitrary game service such as a shooting game, an action game, a role playing game, an adventure game, a race game, a puzzle game, a simulation game, or a table game.

### <Basic Configuration of Information Processing System 1>

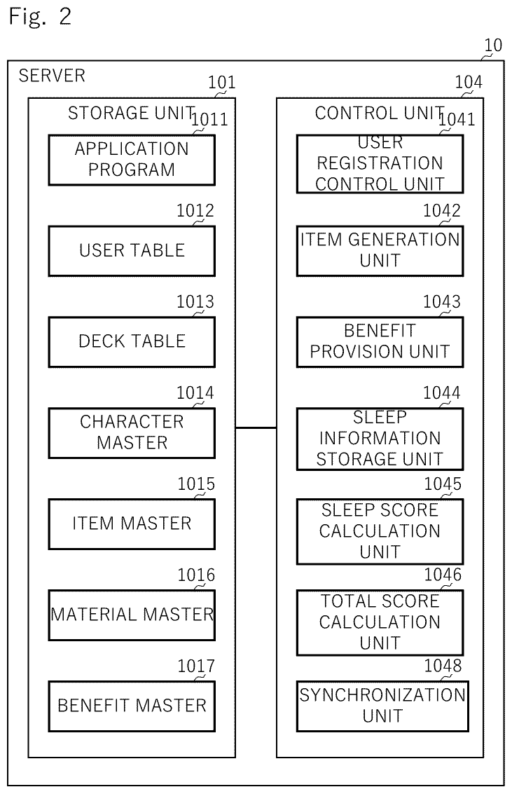

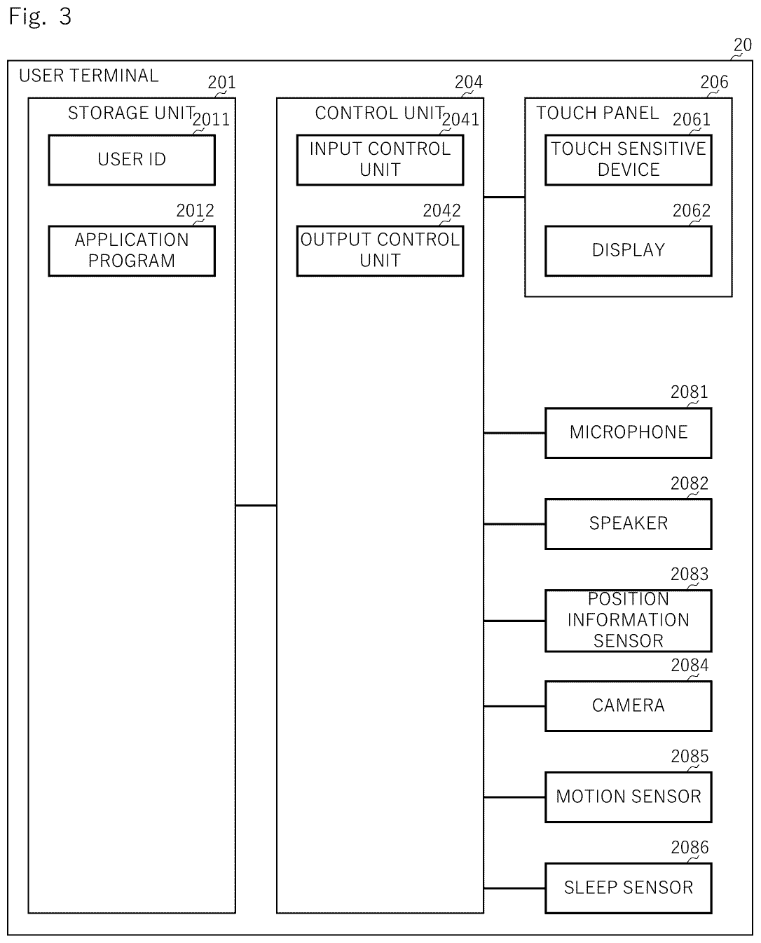

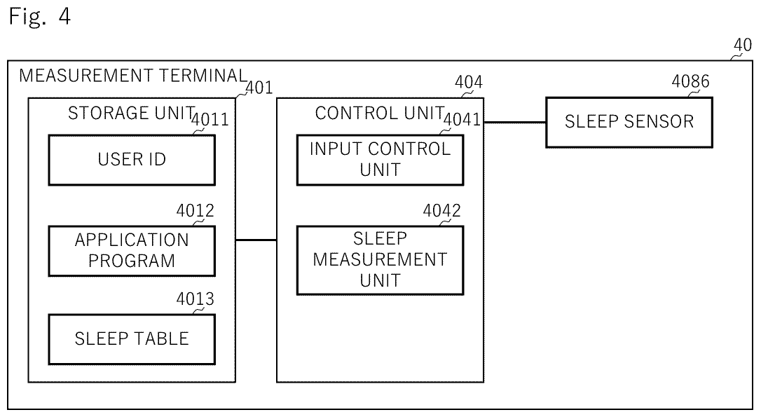

FIG. 1 shows the information processing system 1 according to the present disclosure. The information processing system 1 is configured to include a server 10, a plurality of user terminals 20A, 20B, and 20C, and a plurality of measurement terminals 40A, 40B, and 40C connected to each other via a network N. FIG. 2 is a block diagram illustrating a functional configuration of the server 10. FIG. 3 is a block diagram illustrating a functional configuration of each user terminal 20. FIG. 4 is a block diagram illustrating a functional configuration of each measurement terminal 40. The measurement terminal 40 may be configured to be able to be connected to the user terminal 20 and be connected to the network N via the user terminal 20.

The server 10 is an information processing device that receives information regarding game operations of a user during daytime and a sleep state of the user at nighttime and provides a service of providing a benefit to the user or a character or the like in the game owned by the user.

The user terminal 20 is an information processing device that is operated by the user who uses a service. The user terminal 20 may be, for example, a mobile terminal such as a smartphone or a tablet or a stationary-type personal computer (PC), or a laptop PC. Also, the user terminal 20 may be a wearable terminal such as a head mount display (HMD) or a watch-type terminal.

The measurement terminal 40 is an information processing device for measuring information regarding a sleep state of the user who uses the service at nighttime. Although the measurement terminal 40 is preferably a dedicated terminal mainly for measuring a sleep state of the user at nighttime, the measurement terminal 40 may be, for example, a mobile terminal such as a smartphone or a tablet, a stationary-type personal computer (PC), or a laptop PC. Also, the measurement terminal 40 may be a wearable terminal such as a head mount display (HMD) or a watch-type terminal.

The measurement terminal 40 may be provided in a device that is installed in an everyday sleep environment of the user, such as a pillow, a bed, a blanket, or an alarm clock.

The user terminal 20 may be caused to function as the measurement terminal 40 by causing the user terminal 20 to function as a predetermined application program 2012. The user terminal 20 and the measurement terminal 40 may be the same terminal. The measurement terminal 40 may be a sleep sensor 208 in the user terminal 20. For example, the user terminal 20 may be caused to function as the measurement terminal 40 by causing another application program installed in the user terminal 20 to function.

The measurement terminal 40 may be configured to be able to communicate with the user terminal 20 through wireless communication such as Bluetooth (registered trademark) or WiFi (registered trademark) and may be configured to be able to communicate with the server 10 via the user terminal 20.

Each of the information processing devices is constituted by a computer equipped with a calculation device and a storage device. A basic hardware configuration of the computer and a basic functional configuration of the computer, which is realized by the hardware configuration, will be described below. In regard to each of the server 10, the user terminal 20, and the measurement terminal 40, description overlapping a basic hardware configuration of a computer and a basic functional configuration of the computer, which will be described later, will be omitted.

The configurations and operations of the respective devices are described below.

### <Functional Configuration of Server 10>

FIG. 2 shows a functional configuration realized by the hardware configuration of the server 10. The server 10 includes a storage unit 101 and a control unit 104.

### <Configuration of Storage Unit of Server 10>

The storage unit 101 of the server 10 includes a user table 1012, a deck table 1013, a character master 1014, an item master 1015, a material master 1016, and a benefit master 1017.

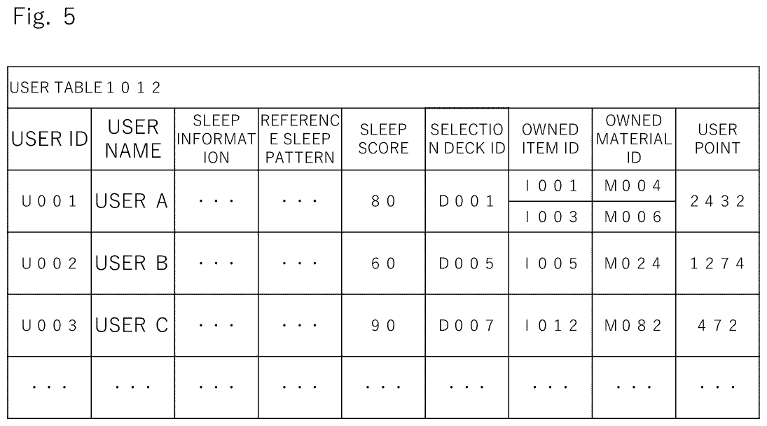

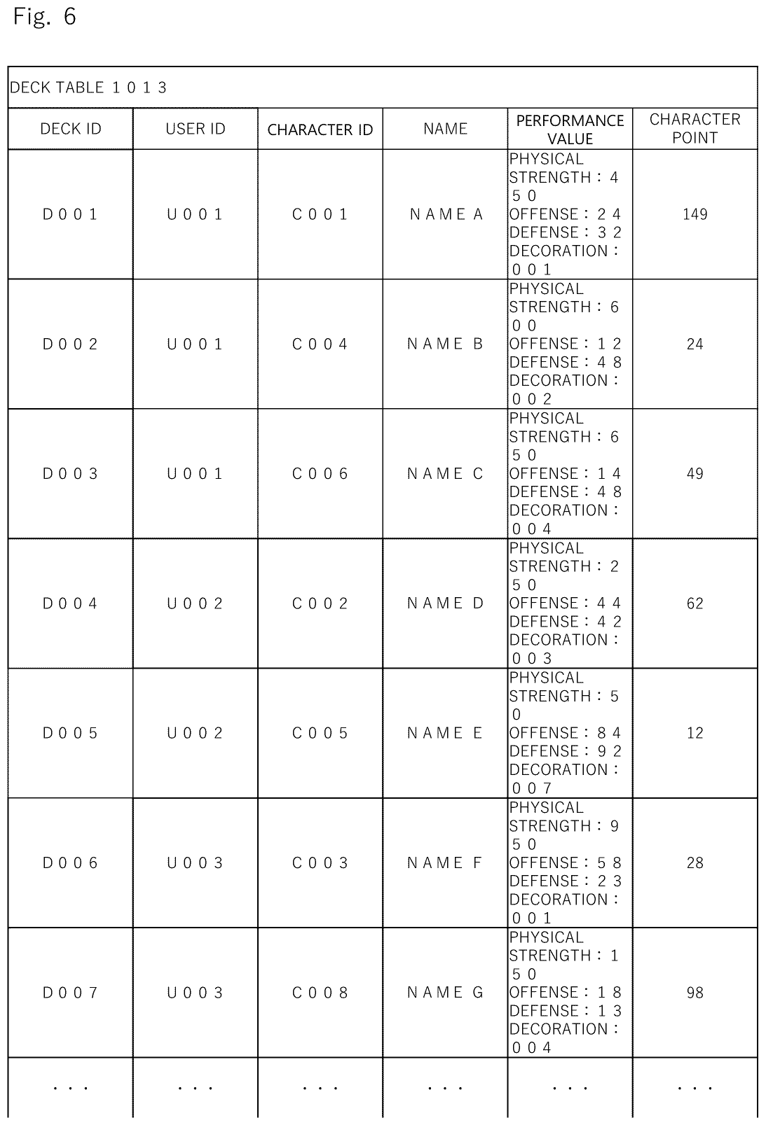

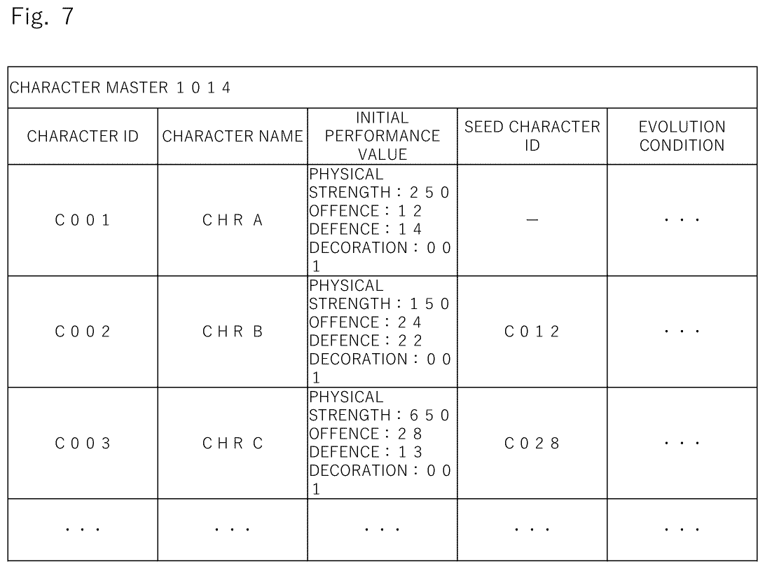

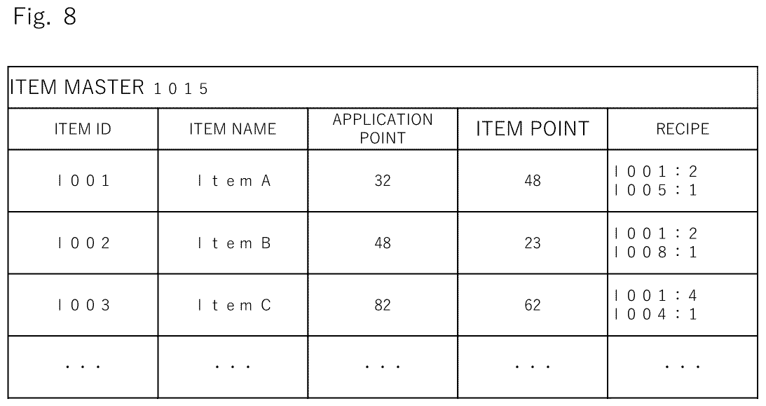

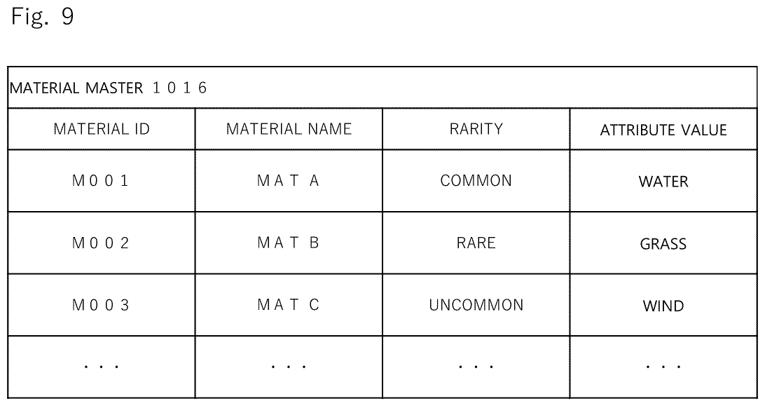

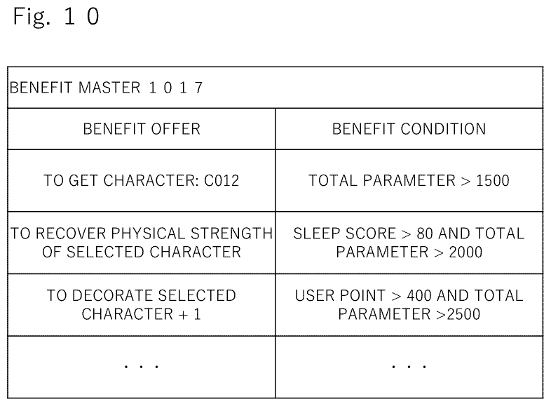

FIG. 5 is a diagram illustrating a data structure of the user table 1012. FIG. 6 is a diagram illustrating a data structure of the deck table 1013. FIG. 7 is a diagram illustrating a data structure of the character master 1014. FIG. 8 is a diagram illustrating a data structure of the item master 1015. FIG. 9 is a diagram illustrating a data structure of the material master 1016. FIG. 10 is a diagram illustrating a data structure of the benefit master 1017.

The user table 1012 is a table that stores and manages information regarding a member user (hereinafter, a user) who uses the service. The information regarding the user is stored as a new record in the user table 1012 by the user performing service utilization registration. In this manner, the user can use the service according to the present disclosure.

The user table 1012 is a table including user IDs as main keys and including columns of user names, play information, sleep information, reference sleep patterns, sleep scores, selection deck IDs, owned item IDs, owned material IDs, and user points.

The user ID is an item that stores user identification information for identifying the user. The user identification information is an item which is a unique value set for each user.

The user name is an item that stores a name of the user.

The play information is an item that stores information (play information) regarding game plays of the user. In the play information, content of game plays (points and parameter values acquired in past game plays) and dates and times when the game plays were performed are stored in an associated manner as history information of the past game plays of the user. In other words, content of a plurality of game plays is stored in the play information for each of the game plays of the user.

The play information may include operations of characters in a game owned by the user, a predetermined operation or provision of items in the game to the characters in the game, achievement of events in the game such as a quest, defeating of enemy characters, and the like.

The sleep information is an item that stores information regarding sleep of the user.

The sleep information may include information regarding a sleep pattern including a bedtime and a wake-up time of the user. Note that the bedtime represents a time of day at which the user goes to sleep and may be, for example, a time of day at which the user goes to bed or a time of day at which the user's consciousness transitions from an awakened state to a state of sleep. In addition, the wake-up time represents a time of day at which the user awakes and may be, for example, a time of day at which the user gets out of bed or a time of day at which the user's consciousness transitions from a state of sleep to an awakened state. In the present embodiment, the pair of the bedtime and the wake-up time from going to sleep to waking up will be referred to as a sleep pattern.

The sleep information may include information regarding quality of sleep of the user. The information regarding quality of sleep refers to a time of a day of each sleep stage in a sleep cycle or a proportion of each sleep stage to the time of day of the sleep. Examples of the information regarding quality of sleep include a time of a day or a proportion of “awakening”, “light sleep”, “deep sleep”, and “REM sleep”, each of which corresponds to a sleep stage of the sleep cycle.

The sleep information may include a history of sleep patterns in a day or a plurality of days during a predetermined period such as one day, a unit of one week, or a unit of one month. Also, the sleep information may include a history of all sleep patterns from the timing when the user starts to use the service to the present.

The reference sleep pattern is an item that stores a predetermined sleep pattern based on a reference bedtime and wake-up time. Since there are individual differences in sleep patterns, a reference sleep pattern in accordance with a constitution of each user may be automatically set on the basis of a sleep history of the user stored in the sleep information.

The reference sleep pattern is not necessarily one fixed sleep pattern, and a plurality of reference sleep patterns may be stored for each day of the week, month, or season, for example. In addition, a plurality of reference sleep patterns may be stored in accordance with weekdays, holidays, national holidays, and the like.

The reference sleep pattern may be set by inputting a bedtime and a wake-up time through conversation between the user and a predetermined character in the game. For example, the reference sleep pattern may be set by allowing the user to input the bedtime and the wake-up time in a form in which the user makes a promise with the predetermined character in the game. In this manner, it is possible to naturally provide motivation for the user to have good sleep habits even in a case where the user is a child.

The sleep score is an item that stores evaluation values related to the sleep of the user.

The selection deck ID is an item that stores a deck ID in the deck table 1013 of a character that the user has currently selected. The user can operate the character in the deck table 1013 associated by the selection character ID for progress of the game and apply an item thereto. Note that, in a case where the number of characters owned by the user is limited to one, the selection deck ID may be omitted.

The owned item ID is an item that stores an item ID or item IDs of a single item or a plurality of items in the game that the user currently owns.

The owned material ID is an item that stores a material ID or material IDs of a single material or a plurality of materials in the game that the user currently owns.

The user point is an item that stores a value of an item point obtained in a case where the user applies an item in the game.

The deck table 1013 is a table for storing characters in the game that the user owns. The user can cause the game to advance by operating the characters in the game that the user himself/herself owns, performing predetermined operations on the characters in the game, applying items in the game, or the like. A group of the characters in the game that the user owns is called a deck in a card game or a party or the like in a roll playing game (RPG).

The user can earn characters in the game by achieving events in the game, such as quests, or satisfying a predetermined condition through a progress of the game, such as defeating of enemy characters. Also, the user may own predetermined characters in the game at the time of the start of the game.

User's owning of characters in the game is expressed by the earned characters in the game being stored in the deck table 1013 with the user ID of the user and character Ids of the characters linked to each other.

The deck table 1013 is a table including deck IDs as main keys and including columns of user IDs, character IDs, names, performance values, and character points.

The deck ID is an item that stores deck identification information for identifying a character that the user owns. The deck identification information is an item in which a unique value is set for each character that the user owns.

The user ID is an item that stores user identification information of the user who owns the deck.

The character ID is an item that stores character identification information of the character in the game that is registered in the deck and is owned by the user.

The name is an item that stores a name of the character in the game that is registered in the deck and is owned by the user. The user can freely set a name for the character in the game that the user himself/herself owns.

The performance value is an item that stores a performance parameter of the character in the game that the user owns. The performance parameter of the character in the game is a parameter such as a status, a physical strength value, a maximum physical strength value, an offensive power, a defensive power, rapidness, a character attribute, or a decoration.

The status is an item that stores a performance parameter related to an activity condition of the character in the game. In a case where the physical strength value of the character in the game is equal to or less than zero, information indicating that the character in the game cannot be active, such as fainting or a death, is stored.

The physical strength value is an item that stores a performance parameter regarding a physical strength of the character in the game. If the character in the game receives a damage, the physical strength value decreases in accordance with content of the damage. Also, if an item in the game is applied to the character in the game or an event in the game such as lodging or resting occurs, the physical value increases (is recovered) in accordance with content of the item and the event.

The maximum physical strength value is an item that stores an upper limit value of the performance parameter regarding the physical strength of the character in the game. The physical strength value of the character in the game can be recovered up to the maximum physical strength value as an upper limit.

The offensive power is an item that stores a performance parameter regarding an offensive power of the character in the game. This is a parameter to calculate a damage that the character in the game will cause damage when the character attacks an opponent character in the game. It is possible to cause a larger damage as the offensive power increases.

The defensive power is an item that stores a performance parameter regarding a defensive power of the character in the game. This is a parameter to calculate a damage that the character in the game receives when the character is attacked by an opponent game character. It is possible to further reduce the damage to be received as the defensive force increases.

The rapidness is an item that stores a performance parameter regarding rapidness of the character in the game. This is a parameter to calculate an attack order when the character in the game attacks an opponent character in the game or receives an attack therefrom. A probability at which the attack order comes early becomes higher as the rapidness is higher than the opponent character in the game.

Also, this may be a parameter to calculate a probability at which an attack is avoided (no damage is received) when an attack is received by the opponent. The probability at which the attack from the opponent is avoided increases as the rapidness is higher than the opponent character in the game.

The character attribute is an item that stores a performance parameter regarding an attribute in the game of the character in the game. The attribute in the game is information prescribing compatibility in attacking or defensing between characters in the game and the compatibility between the characters in the game and environments in the game in the progress of the game, and improves enjoyability of the game by making the progress of the game advantageous or disadvantageous depending on combinations of the compatibilities.

The decoration is an item that stores the type of decoration related to a decoration in the game for a character in the game. The decoration in the game is information that defines a decoration of a character in the game when the user views the character in the game, such as clothes (a shirt, trousers, a skirt, or the like) that the character in the game wears, attached equipment (weapons such as a sword or a spear, protectors such as an armor or a helmet, accessories such as a ring), or other modes, postures, sizes, and the like.

A plurality of decorations are typically prepared in advance as decorations for each character in the game, and a decoration code value or the like in accordance with each decoration is stored. In a case where the user uses the game service in which characters in the game appear, decoration images and decoration objects in accordance with code values are selectively applied to the characters in the game, and the user can further enjoy the game service through the characters in the game decorated by the decoration images and the decoration objects.

The character points are parameters that are added in accordance with a value of an application point of an applied item in the game in a case where a predetermined condition in the game is satisfied (for example, in a case where the item in the game, which will be described later, is applied to a character in the game that the user owns).

The character master 1014 is a table that stores and manages information regarding characters in the game.

The character master 1014 is a table including character IDs as main keys and including columns of character IDs, character names, initial performance values, evolution possible character IDs, and evolution conditions.

The character IDs are items that store character identification information for identifying the characters. The character identification information is an item in which a unique value is set for each character.

The character names are items that store names of the characters (in addition to type names and tribe names, occupation names, job type names, job names, and the like in an RPG game or the like).

The initial performance values are items that store initial values of performance values of the characters in the game. In a case where a character in the game is applied to the user, the performance value of the character in the game is set on the basis of the initial performance value.

The evolution possible character IDs are items that store IDs of predetermined characters called “evolution possible characters”. In a case where the user owns an evolution possible character and evolution conditions, which will be described later, are satisfied, the user can cause the evolution possible character to evolve (also called an occupation change, a job change, a class change, or the like in an RPG game or the like) by performing a predetermined operation.

Specifically, the user selects an evolution possible character stored in the deck table 1013 for the user himself/herself by performing a predetermined operation. Once the user executes an operation of causing evolution in a case where the evolution conditions are satisfied, the character ID of the selected evolution possible character in the deck table 1013 is overwritten by a character ID after the evolution (the character ID linked to the evolution possible character ID). At that time, the performance value and an experience value of the character may also be changed in accordance with the performance value and the experience value of the character before and after the evolution. Also, corresponding display information or decorations of the character may be changed such that the appearance of the character changes before and after the evolution.

The evolution conditions are items that store conditions necessary to cause the evolution possible characters to evolve. Specifically, levels, performance values, experience values, and the like of the evolution possible characters should satisfy predetermined conditions. The user should own predetermined items. In addition, the user should have achieved predetermined events in the game such as quest. In addition, arbitrary conditions such as defeating of predetermined enemy characters, a play time, or the like can be set as the evolution conditions.

The item master 1015 is a table that stores and manages information regarding items in the game.

The user can get items in the game by achieving events in the game such as quest or satisfying a predetermined condition through the progress of the game such as defeating of enemy characters. Also, predetermined items in the game may be owned at the time of a start of the game by regarding the start of the game as a condition.

For the obtained items in the game, the owning of the items in the game by the user is expressed inside the game by the user ID of the user and the item IDs of the items in the game being stored in the user table in a linked manner, for example.

The item master 1015 is a table including item IDs as main keys and including columns of item names, application points, item points, and recipes.

The item IDs are items that store item identification information for identifying items in the game. The item identification information is an item that sets a unique value for each item in the game.

The item names are items that store names (types, tribes, or the like) of the items in the game.

The application points are items that store parameters which are basis of calculation when addition to the character points of the characters in the game is made in a case where items in the game are used (for example, in a case where items in the games are applied to a character in the game).

Specifically, once the user applies an item in the game to a character in the game, processing of adding an application point in accordance with the item in the game to a value of the character point of the character in the game is performed.

Note that processing of subtracting an item point in accordance with an item in the game from a value of the character point of the user may be performed when the user applies the item in the game to the character in the game depending on a combination of the item in the game and the character in the game.

The item points are items that store parameters which are basis of calculation when addition to the user point of the user is made in a case where items in the game are applied to the characters in the game. Specifically, once the user applies an item in the game to a character in the game, processing of adding an item point in accordance with the item in the game to a value of the user point of the user is performed. Note that processing of performing subtraction of an item point in accordance with an item in the game from the value of the user point of the user may be performed when the user applies the item in the game to the character in the game depending on a combination of the item in the game and the character in the game.

The recipes are items that store generation conditions when items in the game are generated from sub-items called “materials”. The user can newly generate and own items in the game by combining a plurality of materials that the user himself/herself owns.

The material master 1016 is a table that stores and manages information regarding the materials in the game.

The user can get materials in the game by achieving events in the game such as quest or through a progress of the game such as defeating of enemy characters. Also, predetermined materials in the game may be owned at the time of a start of the game.

Owning of the materials in the game by the user is expressed inside the game by the obtained materials in the game being stored in the user table with the user ID of the user and the material IDs of the materials in the game linked to each other.

The material master 1016 is a table including material IDs as main keys and including columns of material names, rarities, and attribute values.

The material IDs are items that store material identification information for identifying materials in the game. The material identification information is an item that sets a unique value for each material in the game.

The material names are items that store names (types, tribes, or the like) of the materials in the game.

The rarities are items that store rarities of the materials in the game. Rarity values such as “super rare”, “rare”, and “common”, for example, are stored in accordance with levels of difficulty in getting the materials.

The attribute values are items that store attribute values of the materials in the game. The attribute values are items that are used as conditions when predetermined items in the game are generated in recipe items in the item master.

The benefit master 1017 is a table that stores benefits to be provided to the user.

The benefit master 1017 is a table including columns of benefit offers and benefit conditions.

The benefit offers are items that stores offers of benefits to be provided to the user.

The offer of benefits to be provided to the user may include an offer of benefits provided to the user. For example, the offer of benefits may include an offer that the user will newly get a character in the game. The offer of benefits may include an offer that an item in the game or a material in the game will be provided to the user. The offer of benefits may include an offer that an item in the game will be generated from predetermined materials in the game that the user owns.

The offer of benefits to be provided to the user may include the offer of benefits to be provided to a character that the user owns. For example, the offer of benefits may include an offer that a performance value of the character in the game that the user owns will be enhanced or weakened.

Also, the offer of benefits may include an offer that a decoration of the character in the game that the user owns will be changed.

The offers of benefits to be applied to the user may include an offer that a character in the game (a predetermined evolution possible character) that the user owns will be caused to evolve.

The benefit conditions are items that store conditions when benefits are provided to the user. As a benefit condition in the present disclosure, a range of a total score is stored. In other words, whether or not a value of the total score meets the condition of benefits is determined, and in a case where the condition is met, the benefit offers linked to the benefit condition is provided to the user.

A benefit A is provided in a case where the total score falls between 11 to 100 points, and a benefit B is provided in a case where the total score falls between 101 to 200 points, for example.

Note that the benefit conditions are not necessarily exclusive conditions for each benefit offer and may be a plurality of overlapping conditions. In other words, one total score may satisfy a plurality of benefit conditions. At this time, a plurality of benefit offers in accordance with the plurality of benefit conditions may be provided to the user in accordance with the value of the one total score.

Also, only one benefit offer with a high priority may be provided to the user from among one or a plurality of benefit offers by setting priorities for the benefit offers.

### <Configuration of Control Unit of Server 10>

The control unit 104 of the server 10 includes a user registration control unit 1041, an item generation unit 1042, a benefit provision unit 1043, a sleep information storage unit 1044, a sleep score calculation unit 1045, a total score calculation unit 1046, and a synchronization unit 1048. Each functional unit of the control unit 104 is realized by executing an application program 1011 stored in the storage unit 101.

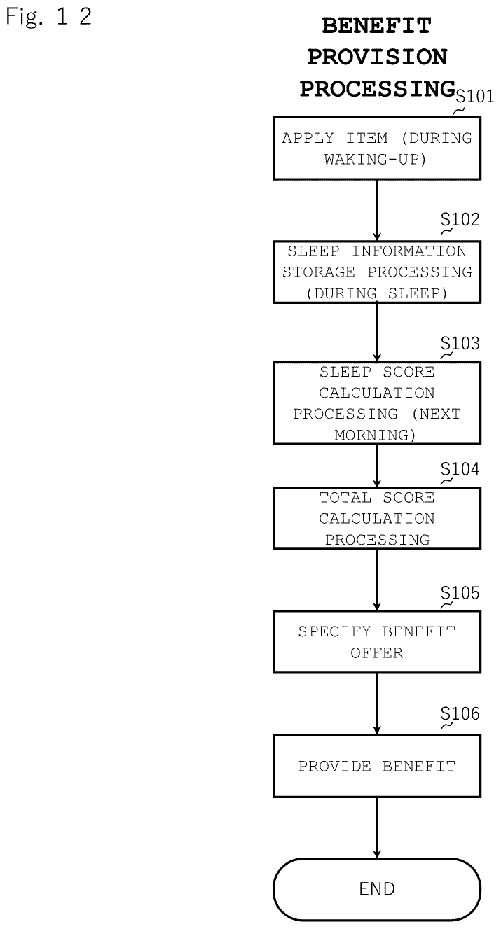

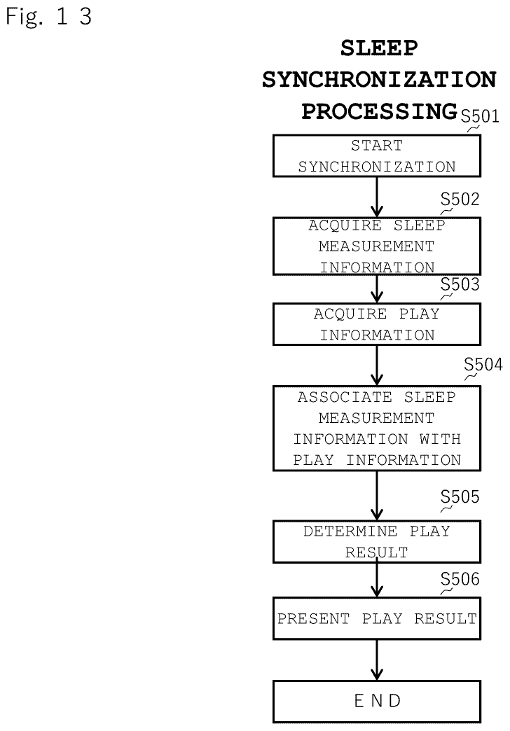

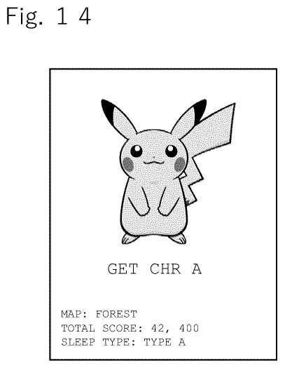

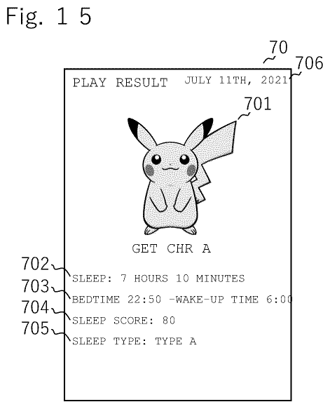

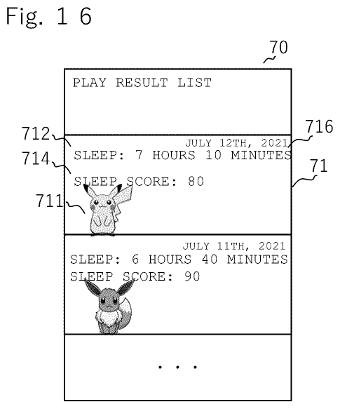

FIG. 12 is a flowchart illustrating operations in benefit provision processing. FIG. 13 is a flowchart illustrating operations in sleep synchronization processing. FIG. 14 is an example of a screen illustrating operations in the benefit provision processing. FIG. 15 is an example of a screen presenting a play result in a first presentation mode in the sleep synchronization processing. FIG. 16 is an example of a screen presenting a play result in a second presentation mode in the sleep synchronization processing.

The user registration control unit 1041 performs processing of storing, in the user table 1012, information of a user who desires to use the service according to the present disclosure.

For information regarding a user name to be stored in the user table 1012, the user opens a web page administered by a service provider from an arbitrary information processing terminal, inputs the user name to a predetermined input form, and transmits it to the server 10. The user registration control unit 1041 of the server 10 stores the received user name as a new record in the user table 1012, and user registration is then completed. In this manner, the user, the information of which has been stored in the user table 1012, can start to use the service.

The service provider may conduct a predetermined examination and limit service usability of the user before the user registration control unit 1041 registers the user information in the user table 1012.

The user ID may be an arbitrary character sequence or number with which the user can be identified, and an arbitrary character sequence or number that the user desires may be set, or the user registration control unit 1041 of the server 10 may automatically set an arbitrary character sequence or number.

The item generation unit 1042 executes item generation processing. Details will be described below.

The benefit provision unit 1043 executes benefit provision processing. Details will be described below.

The sleep information storage unit 1044 executes sleep information storage processing. Details will be described below.

The sleep score calculation unit 1045 executes sleep score calculation processing. Details will be described below.

The total score calculation unit 1046 executes total score calculation processing. Details will be described below.

The synchronization unit 1048 executes sleep synchronization processing. Details will be described below.

### <Functional Configuration of User Terminal 20>

A functional configuration that a hardware configuration of the user terminal 20 realizes is illustrated in FIG. 3. The user terminal 20 includes a storage unit 201, a control unit 204, a touch panel 206, a touch sensitive device 2061, a display 2062, a microphone 2081, a speaker 2082, a position information sensor 2083, a camera 2084, a motion sensor 2085, and a sleep sensor 2086.

The sleep sensor 2086 may be various devices that detects various states of the user terminal 20. The sleep sensor 2086 may include, for example, a posture sensor (an acceleration sensor or a gyroscope sensor) that detects a posture or an inclination of the terminal itself, an eye-gaze sensor that detects a direction of a line of sight of the user, a photosensor that detects peripheral brightness, or an infrared sensor that detects motions of the user. Also, the sleep sensor 2086 may be a microphone that collects sound in the surroundings of the user terminal 20, a humidity sensor that detects humidity in the surroundings of the user terminal 20, a geomagnetic sensor that detects a magnetic field at a location where the user terminal 20 resides, or the like.

The sleep sensor 2086 may detect various kinds of information using the sensor functions described above. For example, the sleep sensor 2086 may detect the number of steps walked by the user who owns the user terminal 20 using the function of the acceleration sensor. Also, the sleep sensor 2086 may detect operation information indicating whether the user terminal 20 is operating or standing still, for example, using the function of the acceleration sensor every specific time or every time the user terminal 20 operates. The sleep sensor 2086 can transmit sensing data detected as described above to the control unit 204.

Also, the sleep sensor 2086 may be an information processing terminal that the user can wear (so-called a wearable terminal) that is provided as a device separated from the user terminal 20 like a watch-type terminal or a ring-type terminal and is communicably connected to the user terminal 20. At this time, the sleep sensor 2086 can detect biological information of the user and transmit the biological information to the user terminal 20. The sleep sensor 2086 can determine a heart rate of the user by photo plethysmography, for example, and transmit the heart rate as sensing data to the control unit 204. Note that the sensing data detected by the sleep sensor 2086 is not limited thereto, and biological information regarding sleep of the user, for example, brain waves, breathing, a pulse, body motion, or the like may be detected.

### <Configuration of Storage Unit of User Terminal 20>

The storage unit 201 of the user terminal 20 stores a user ID 2011 for identifying the user who uses the user terminal 20 and an application program 2012.

The user ID is an account ID of the user. The user transmits the user ID 2011 from the user terminal 20 to the server 10. The server 10 identifies the user on the basis of the user ID 2011 and provides the service according to the present disclosure to the user. Note that the user ID includes information such as a session ID that is temporarily applied from the server 10 to identify the user who uses the user terminal 20.

The application program 2012 may be stored in the storage unit 201 in advance or may be configured to be downloaded from a web server or the like administered by the service provider via the communication IF. The application program 2012 includes an interpreter-type programming language executed on a web browser application stored in the user terminal 20.

### <Configuration of Control Unit of User Terminal 20>

The control unit 204 of the user terminal 20 includes an input control unit 2041 and an output control unit 2042. The functional units of the input control unit 2041 and the output control unit 2042 are realized by the control unit 204 executing the application program 2012 stored in the storage unit 201.

The input control unit 2041 of the user terminal 20 acquires user's operation content on the touch sensitive device 2061 of the touch panel 206, a sound input to the microphone 2081, and information output from input devices such as the position information sensor 2083, the camera 2084, the motion sensor 2085, and the sleep sensor 2086 and executes various kinds of processing. The input control unit 2041 of the user terminal 20 executes processing of transmitting the information acquired from the input devices to the server 10 along with the user ID 2011.

The output control unit 2042 of the user terminal 20 receives user's operations on the input devices and information from the server 10 and executes processing of controlling display content of the display 2062 and sound output content of the speaker 2082.

### <Functional Configuration of Measurement Terminal 40>

A functional configuration that the hardware configuration of the measurement terminal 40 realizes is illustrated in FIG. 4. The measurement terminal 40 includes a storage unit 401, a control unit 404, and a sleep sensor 4086.

The sleep sensor 4086 is a sensor that is similar to the sleep sensor 2086 that the user terminal 20 has, and description thereof will thus be omitted.

### <Configuration of Storage Unit of Measurement Terminal 40>

The storage unit 401 of the measurement terminal 40 stores a user ID 4011 for identifying a user who uses the measurement terminal 40, an application program 4012, and a sleep table 4013.

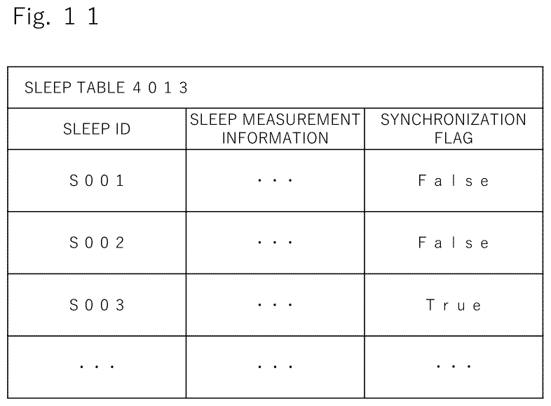

FIG. 11 is a diagram illustrating a data structure of the sleep table 4013.

The user ID is an account ID of the user. The user transmits the user ID 4011 from the measurement terminal 40 to the server 10. The server 10 identifies the user on the basis of the user ID 4011 and provides the service according to the present disclosure to the user. Note that the user ID includes information such as a session ID temporarily applied from the server 10 to identify the user who is using the measurement terminal 40.

Note that the user ID 4011 stored in the measurement terminal 40 is not necessarily the same as the user ID 2011 stored in the user terminal 20 and may be any ID as long as it is possible to identify that the same user uses the information processing devices by being associated with the user ID 2011 using a table or the like. Note that the user ID 4011 is not essential and a configuration in which association with a predetermined user is made in the sleep synchronization processing, which will be described later, may be employed.

The application program 4012 may be stored in the storage unit 401 in advance or may be configured to be downloaded from a web server or the like administered by the service provider via the communication IF. The application program 4012 includes an interpreter-type programming language executed on a web browser application stored in the measurement terminal 40.

The sleep table 4013 is a table that stores and manages information regarding sleep of the user measured by the sleep sensor 4086 of the measurement terminal 40.

The sleep table 4013 is a table including sleep IDs as main keys and including columns of sleep measurement information and synchronization flags.

The sleep ID is sleep identification information for identifying sleep information. The sleep identification information is an item for which a unique value is set for each sleep.

The sleep measurement information is an item that stores information regarding sleep of the user for each sleep. In other words, one record of the information regarding sleep is stored in the item of the sleep measurement information for each sleep. Information for each sleep is information regarding each sleep from a bedtime to a wake-up time.

The sleep measurement information may include information equivalent to the sleep information in the user table 1012. Specifically, the sleep measurement information may include information regarding a sleep pattern indicating a bedtime and a wake-up time of the user. The sleep measurement information may include information regarding quality of sleep of the user. The sleep measurement information may include information regarding a date when the sleep information has been measured. Specifically, the sleep measurement information may include information regarding a date and a time (a start date and time, for example) when the sleep measurement processing is executed.

The synchronization flag is an item that stores whether or not the sleep measurement information has been synchronized. Information indicating that the sleep measurement information has not been synchronized, such as an empty value, a null value, or another value is stored in a record with which the sleep measurement information has not been synchronized. In a case where the sleep measurement information has been synchronized, information indicating that the sleep measurement information has been acquired and synchronized is stored.

### <Configuration of Control Unit of Measurement Terminal 40>

The control unit 404 of the measurement terminal 40 includes an input control unit 4041 and a sleep measurement unit 4042. The functional units of the input control unit 4041 and the sleep measurement unit 4042 are realized by the control unit 404 executing the application program 4012 stored in the storage unit 401.

The input control unit 4041 of the measurement terminal 40 acquires information output from the input devices such as the sleep sensor 4086 and executes various kinds of processing. The input control unit 4041 of the measurement terminal 40 executes processing of transmitting the information acquired from the input devices to the server 10 along with the user ID 4011.

The sleep measurement unit 4042 executes sleep measurement processing. Details will be described below.

### <Operations of Information Processing System 1>

Hereinafter, each process of the information processing system 1 will be described with reference to FIGS. 12 and 13.

First, the control unit 104 of the server 10 receives a request including a user ID from the user terminal 20 and starts to provide various services.

In the present disclosure, a game service that uses, as motifs, cooked foods (hereinafter, foods) as items in the game and ingredients, seasonings, and the like as materials in the game will be described.

### <Item Generation Processing>

Item generation processing is processing of generating items in the game from materials in the game.

### <Overview of Item Generation Processing>

The item generation processing is a series of processes in which materials in the game that the user owns are consumed and the user gets new items in the game.

### <Details of Item Generation Processing>

The user operates the touch panel 206 of the user terminal 20 of the user himself/herself and transmits the user ID 2011 of the user himself/herself and an inquiry request for an item in the game that can be generated to the server 10.

Once the item generation unit 1042 of the server 10 receives a request from the user terminal 20, the item generation unit 1042 searches the user table 1012 using the received user ID as a search key and acquires user's owned material IDs. The item generation unit 1042 of the server 10 searches the material master 1016 using the owned material IDs as search keys and acquires rarities, attribute values, and the like of the materials owned by the user. The item generation unit 1042 of the server 10 refers to items of recipes in the item master 1015 from information based on the materials and the like in the game that the user owns, searches item IDs of items in the game that the user can generate, and acquires the item IDs. In a case where the user owns materials in the game stored in the items of the recipes in the item master 1015 (the types and numbers of the materials), for example, the item generation unit 1042 of the server 10 determines that the items in the game are items in the game that can be generated. Note that the recipes may define generation conditions of the items in the game on the basis of conditions such as attribute values and rarities of the materials in the game.

The item generation unit 1042 of the server 10 can acquire the item IDs of the foods that can be cooked from the ingredients that the user owns.

The item generation unit 1042 of the server 10 transmits the item IDs of the items in the game that the user can generate to the user terminal 20. The user terminal 20 outputs the items in the game that the user can generate to the display 2062 of the user terminal 20 and displays them on the display 2062 on the basis of the item IDs received from the server 10.

The user can thus view and check the list of the foods that can be cooked.

The user operates the touch panel 206 of the user terminal 20 and selects an item in the game that the user desires to generate from the items in the game displayed on the display 2062. Once the user selects the item in the game, the control unit 204 of the user terminal 20 transmits the item ID of the selected item in the game to the server 10.

The user can thus select a food that the user desires to cook.

The item generation unit 1042 of the server 10 searches the item master 1015 on the basis of the received item ID and acquires information regarding the recipe. The item generation unit 1042 of the server 10 deletes the material in the game necessary to generate the item in the game from the owned material IDs in the record related to the user in the user table 1012 on the basis of generation conditions defined in the information regarding the recipe. In other words, the user consumes the materials in the game that the user himself/herself owns to generate the item in the game. Note that content processed in the generation of the item in the game is not limited to the consumption of the materials in the game and arbitrary processing may be performed in accordance with generation conditions.

Next, the item generation unit 1042 of the server 10 adds the received item ID to a column of owned item IDs of the record regarding the user in the user table 1012. In other words, the user gets the selected item in the game.

The user can thus newly get the food by consuming the materials in the game such as ingredients and seasonings.

Note that a configuration in which when the user gets materials in the game without selecting any item in the game that can be selected, an item in the game that satisfies generation conditions is automatically generated and the user gets the item may be employed. Note that a configuration in which a predetermined item in the game is generated randomly or in accordance with a predetermined probability and the user gets the item in a case where a plurality of items in the game that can be generated are present may be employed. The user can thus get a new item in the game without the complicated operation of selecting an item in the game that can be generated, and the user can further enjoy the game service.

In this case, once the user performs operations in the game and gets ingredients and seasonings, the user can automatically get foods that can be cooked from the ingredients and the seasonings.

### <Benefit Provision Processing>

Benefit provision processing is processing of receiving information regarding game operations when the user is awake (during daytime, for example) and sleep states when the user sleeps (at nighttime, for example) and providing benefits to the user. The user can acquire the benefits through the benefit provision processing. Note that the benefits given to the user include benefits to characters in the game that the user owns, benefits given to a team in the game, a group in the game, or the like to which the user belongs, and the like, and include arbitrary benefits to arbitrary objects in the game, entities in the game, and the like associated with the user.

Hereinafter, details of the benefit provision processing will be described by using the flowchart in FIG. 12 and an example of a display screen in FIG. 14.

### <Overview of Benefit Provision Processing>

The benefit provision processing is processing of providing benefits (bonuses) to the user in accordance with items applied to characters and a sleep score.

Specifically, a total score is calculated on the basis of a character point calculated on the basis of the items applied to the character and sleep score. Benefits in accordance with the total score is determined, and the determined benefits are provided to the user. The user can thus get the benefits.

### <Details of Benefit Provision Processing>

Details of the benefit provision processing will be described below.

In Step S101, the user executes an operation using items in a time zone other than a bedtime zone, such as during daytime. Specifically, an operation of applying items to a character that the user is currently selecting is executed. The user can repeatedly execute the operation using items. Note that the server 10 and the user terminal 20 may limit operations so as not to receive any operations from the user even if the user is awake during the bedtime zone. It is thus possible to motivate the user to have good sleep habits with higher quality.

The user operates the touch panel 206 of the user terminal 20 of the user himself/herself and transmits the user ID 2011 of the user himself/herself and an inquiry request for items in the game that the user owns to the server 10.

Once the benefit provision unit 1043 of the server 10 receives the request from the user terminal 20, the benefit provision unit 1043 searches the user table 1012 using the received user ID as a search key, acquires owned item IDs of the user, and transmits the owned item IDs to the user terminal 20.

The user terminal 20 outputs items in the game that the user can apply to the display 2062 of the user terminal 20 and displays them on the display 2062 on the basis of the item IDs received from the server 10.

The user can thus view and check the list of foods that can be applied and check what kinds of foods can be applied to the character.

The user operates the touch panel 206 of the user terminal 20, selects items in the game that the user desires to apply from among the items in the game displayed on the display 2062, and provides an instruction to use the items in the game. Once the user selects the item in the game, the control unit 204 of the user terminal 20 transmits the item ID of the selected item in the game to the server 10.

The user can thus select a food that the user desires to apply.

The benefit provision unit 1043 of the server 10 searches the item master 1015 on the basis of the received item IDs and acquires applied point and item points.

The benefit provision unit 1043 of the server 10 adds the applied point to character point of the character that the user is selecting. Specifically, the benefit provision unit 1043 of the server 10 makes a reference to the deck table 1013 on the basis of a deck ID stored in a selection deck ID of the record regarding the user in the user table 1012 and specifies a record of the character that the user is selecting. Then, a value of the applied point acquired is added to the character point stored in the record regarding the character. In this manner, a parameter regarding the applied point of the character that the user owns is generated. In other words, a parameter regarding the user is generated.

The benefit provision unit 1043 of the server 10 may cause a decoration of the character to change in accordance with the character point. For example, an image of the character displayed on the display 2062 of the user terminal 20 may be displayed with a larger size in accordance with an increase in character point. It is thus possible to provide a performance in which the character increases in size to the user by applying the food to the character. The user can further enjoy the game service.

The benefit provision unit 1043 of the server 10 adds the acquired item point to the user point of the user.

Specifically, the benefit provision unit 1043 of the server 10 adds the value of the acquired item point to the user point stored in the record regarding the user in the user table 1012.

### <Sleep Information Storage Processing>

In Step S102, the sleep information storage unit 1044 of the server 10 executes sleep information storage processing of storing sleep information of the user who is sleeping.

The user terminal 20 may receive an input operation indicating that the user will go to bed and use the input operation as a trigger to start the sleep information storage processing. The input operation indicating that the user will go to bed may be, for example, an operation of interrupting the game or an operation of putting the character to sleep (stroking the character via the touch panel 206, turning off the light that the character is using, or the like). Also, the user terminal 20 can also constantly execute sensing using the sleep sensor 2086 and detect the user's going to bed from sensing data (motions of the terminal, or brightness or sound in the surroundings, for example). Also, the user may be encouraged to go to bed by causing the character to look sleepy or turning the game world to night when a reference bedtime, which will be described later, approaches.

The control unit 204 of the user terminal 20 provides an instruction to execute sensing to the sleep sensor 2086. Specifically, the sleep sensor 2086 senses motions of the user terminal 20 and biological information such as a heart rate of the user. The control unit 204 of the user terminal 20 acquires sensing data from the sleep sensor 2086.

The control unit 204 of the user terminal 20 transmits the sensing data sent from the sleep sensor 2086 to the server 10. The sleep information storage unit 1044 of the server 10 stores a sleep pattern indicating a bedtime and a wake-up time in a column of the sleep information of the record of the user in the user table 1012 on the basis of the sensing data.

Information regarding sleep quality of the user may be included in the sleep information. At this time, the user terminal 20 calculates the information regarding sleep quality of the user from the biological information detected by the sleep sensor 2086. The information regarding sleep of the user may be calculated by employing a known function of analyzing sleep in an existing multifunctional device that functions as the user terminal 20.

The sleep information storage unit 1044 of the server 10 stores the information regarding the sleep quality in the column of the sleep information of the user in the user table 1012.

The sleep information storage unit 1044 of the server 10 may be configured to execute the sleep information storage processing only once in a predetermined period of time. Note that the control unit 204 of the user terminal 20 may be configured to transit a sleep pattern to the server 10 only once in the predetermined period of time, or the sleep information storage unit 1044 of the server 10 may be configured to store a sleep pattern in the column of the sleep information in the record of the user in the user table 1012 from among a plurality of sleep patterns received from the user terminal 20 only once in a predetermined period of time.

For example, the sleep information storage unit 1044 of the server 10 may store only one sleep pattern from among a plurality of sleep patterns in which bedtimes have been detected from 5:00 AM to 4:59 AM in the next morning in the column of the sleep information in the record of the user in the user table 1012.

Specifically, the sleep information storage unit 1044 of the server 10 stores a sleep pattern that has been detected for the first time after 5:00 AM in the column of the sleep information in the record of the user in the user table 1012 and does not store sleep patterns that have been detected after the sleep and before 4:59 AM in the next morning.

For example, the sleep information storage unit 1044 of the server 10 may store only a sleep pattern showing the longest sleep time from among the plurality of sleep patterns in which bedtimes have been detected from 5:00 AM to 4:59 AM in the next morning in the column of the sleep information of the record of the user in the user table 1012.

In another case, the sleep information storage unit 1044 of the server 10 may add up a plurality of sleep patterns from among a plurality of sleep patterns in which bedtimes have been detected from 5:00 AM to 4:59 AM in the next morning and store the added sleep patterns in the column of the sleep information in the record of the user in the user table 1012. For example, it is conceivable that the sleep information storage unit 1044 of the server 10 adds up sleep times of the plurality of sleep patterns and stores the sleep times in the column of the sleep information in the record of the user in the user table 1012.

It is thus possible to limit the number of times the sleep measurement information is stored in the predetermined period of time to only once. Specifically, it is possible to limit the number of times the sleep measurement information can be stored a day for each user to only once.

### <Sleep Score Calculation Processing>

In Step S103, the sleep score calculation unit 1045 of the server 10 executes sleep score calculation processing of calculating a sleep score on the basis of the sleep information in the next morning of the day when the user applies items. In other words, the sleep score is calculated and determined from the sleep information.

The sleep score calculation processing may be executed in an arbitrary time zone other than the bedtime zone, such as during daytime. The sleep score calculation processing may be executed when the user operates the user terminal 20 and activates or operates an application or the like related to the game service according to the present disclosure. The sleep score calculation unit 1045 of the server 10 may execute the sleep score calculation processing in a case where a wake-up time of the user is detected.

The sleep score calculation unit 1045 of the server 10 determines whether or not the sleep pattern included in the sleep information stored in the sleep information in the user table 1012 satisfies a reference sleep pattern.

The sleep score calculation unit 1045 of the server 10 searches the user table 1012 using the user ID received from the user terminal 20 as a search key and acquires the sleep information and the reference sleep pattern.

Hereinafter, the present disclosure will be described on the assumption that the reference bedtime is 21:00 and the reference wake-up time is 7:00.

The reference sleep pattern is set on the basis of the reference bedtime and the reference wake-up time (hereinafter, the reference bedtime and/or the reference wake-up time may be described as a reference clock time) and may have a width of a time zone within a range in which the shortest sleep time is secured. A width is given within a range of 30 minutes before the reference clock time and 15 minutes after the reference clock time. The width of the reference clock time may differ before the reference clock time and after the reference clock time. For example, the width of the time zone before the reference clock time is set to be longer than the width of the time zone after the reference clock time from the viewpoint of recommending to go to bed early and get up early. In addition, a setting of the minimum sleep time may be changed according to the age of the user. The user or the administrator of the game service can set 7 hours for adults and 10 hours for children, for example.

In a case where a bedtime is included in the reference bedtime (time zone) and a wake-up time is also included in the reference wake-up time (time zone) in a sleep pattern A, the sleep score calculation unit 1045 of the server 10 determines that the sleep pattern A satisfies the reference sleep pattern.

In a case where a bedtime is 21:00 and satisfies the reference bedtime while a wake-up time is after 7:15 and does not satisfy the reference wake-up time in a sleep pattern B, the sleep score calculation unit 1045 of the server 10 determines that the sleep pattern B does not satisfy the reference sleep pattern.

In a case where a wake-up time is between 7:00 to 7:15 and satisfies the reference wake-up time while a bedtime is after 21:15 and does not satisfy the reference bedtime in a sleep pattern C, the sleep score calculation unit 1045 of the server 10 determines that the sleep pattern C does not satisfy the reference sleep pattern.

As described above, the sleep score calculation unit 1045 of the server 10 determines whether the sleep pattern satisfies the reference sleep pattern.

In a case where the sleep score calculation unit 1045 of the server 10 determines that the reference sleep pattern is not satisfied, the sleep score calculation unit 1045 of the server 10 calculates the sleep score as 10 points.

In the sleep score calculation unit 1045 of the server 10 determines that the reference sleep pattern is satisfied, the sleep score calculation unit 1045 of the server 10 determines whether the sleep pattern continuously satisfies the reference sleep pattern for a predetermined period of time or more on the basis of the sleep information.

In a case where it is determined that the sleep pattern based on the sleep information does not satisfy the reference sleep pattern for the predetermined period of time or more, the sleep score calculation unit 1045 of the server 10 calculates the sleep score as 40 points.

In a case where it is determined that the sleep pattern based on the sleep information satisfies the reference sleep pattern for the predetermined period of time or more, the sleep score calculation unit 1045 of the server 10 calculates the sleep score as 70 points.

In a case where the sleep time based on the sleep information is longer than the predetermined time, the sleep score calculation unit 1045 of the server 10 may determine that the reference sleep pattern is satisfied. The sleep score calculation unit 1045 of the server 10 may add a greater sleep score as the sleep time based on the sleep information is longer. In another case, the sleep score calculation unit 1045 of the server 10 may calculate the sleep score on the basis of the sleep time based on the sleep information.

The sleep score calculation unit 1045 of the server 10 may determine that the reference sleep pattern is satisfied in a case where a deep sleep time based on the sleep information is longer than a predetermined time and in a case where the number of times deep sleep happens is greater than a predetermined number of times. The sleep score calculation unit 1045 of the server 10 may add a larger sleep score as the length of the time of the deep sleep based on the sleep information is longer and as the number of times the deep sleep happens is greater. In another case, the sleep score calculation unit 1045 of the server 10 may calculate the sleep score on the basis of a time during which deep sleep happens or the number of times the deep sleep happens based on the sleep information.

The sleep score calculation unit 1045 of the server 10 may add or subtract the sleep score in accordance with a degree of deviation of the sleep pattern based on the sleep information from the reference sleep pattern. In other words, the sleep score calculation unit 1045 may perform calculation on the assumption that the sleep score is higher as the sleep pattern based on the sleep information is closer to the reference sleep pattern and the sleep score is lower as the deviation from the reference sleep pattern becomes greater. It is thus possible to motivate the user for good sleep habits based on a regular sleep pattern.

The user or the administrator of the game service may be able to set an arbitrary predetermined period of time. For example, the predetermined period of time may be three days, one week, or the like.

The user may set the predetermined period of time by providing an input through conversation with a predetermined character in the game. For example, the user may set the predetermined period of time by providing an input in the form in which the user promises the predetermined period of time with the predetermined character in the game. In this manner, it is possible to naturally provide a motivation for good sleep habits to the user even in a case where the user is a child.

Also, in a case where the sleep information includes information regarding sleep quality, the sleep score calculation unit 1045 of the server 10 may add or subtract the sleep score on the basis of an indicator from which it is possible to know whether the sleep is good, on the basis of a sleep stage. The sleep score may be added or subtracted on the basis of an arbitrary algorithm to weighting to each sleep stage.

Addition to or subtraction from the sleep score may be performed in accordance with the amount of activity, meals, a location, humidity, an operation time of the user terminal, an operation time zone, and the like of the user. In a case where a location of a highly evaluated hotel (a five-star hotel or the like) is detected as position information of the user terminal, for example, addition may be made to the sleep score on the assumption that good sleep can be obtained. In addition, the sleep score may be reduced if the user terminal is operated after the reference bedtime, for example.

The sleep score calculation unit 1045 of the server 10 stores the calculated sleep score in the column of the sleep score of the record regarding the user in the user table 1012.

### <Total Score Calculation Processing>

In Step S104, the total score calculation unit 1046 of the server 10 calculates a total score on the basis of a character point and a sleep score.

The total score is a value calculated on the basis of the character point and the sleep score. In other words, the total score is a value calculated and determined by the play information and the sleep information. The total score is calculated as a product of a sleep score on the day (the sleep score when the user woke up) and a character point on the previous day (the character point when the user went to bed), for example. The total score may be calculated on the basis of a statistical value (such as an average value, a median, a most frequent value, a maximum value, or a minimum value) of sleep scores over a plurality of days and a statistical value of character points over the plurality of days.

The total score calculation unit 1046 of the server 10 searches the user table 1012 using the user ID received from the user terminal 20 as a search key and acquires the character point and the sleep score.

The total score calculation unit 1046 of the server 10 computes a product of the acquired character point and sleep score and calculates the total score. Note that the total score may be calculated on the basis of an arbitrary algorithm such as a sum of the character point and the sleep score.

The total score calculation unit 1046 of the server 10 stores the calculated total score in the column of the total score in the record regarding the user in the user table 1012.

Note that the total score calculation unit 1046 of the server 10 may be configured to calculate the total score on the basis of one of the values, namely either the character point or the sleep score.

In Step S105, the benefit provision unit 1043 of the server 10 specifies the benefit offer to be provided on the basis of the total score.

The benefit provision unit 1043 of the server 10 searches a benefit condition in the benefit master 1017 on the basis of the calculated total score and the like.

Specifically, a record of the benefit condition that satisfies a condition such as a calculated total score or the like is specified. Then, the benefit offer stored in the record is acquired. Note that as the benefit condition in the benefit master 1017, an arbitrary condition based on the character point, the sleep score, or the like can be set instead of the total score.

Note that although the configuration in which the benefit offer is specified on the basis of the total score has been described on the present disclosure, a configuration in which the benefit offer is specified from the character point and the sleep score without calculating the total score may also be employed.

In Step S106, the benefit provision unit 1043 of the server 10 provides the benefit to the user or the character or the like in the game that the user owns on the basis of the acquired benefit offer.

The benefit provision unit 1043 of the server 10 provides the benefit to the user on the basis of the information regarding the acquired benefit offer. As an example of the benefit, the user can get a new character in the game. An example of an exemplary screen illustrating operations in the benefit provision processing is illustrated in FIG. 14. An image of the obtained character, the value of the total score, and a text regarding a “sleep type” indicating the sleep state of the user determined on the basis of the sleep score or the total score are also displayed on the display 2062 of the user terminal 20. As the sleep type, a text such as “type A”, “type B”, or “type C” expressing the sleep state of the user is displayed. Note that the sleep information in the present disclosure includes, in addition to the sleep score and the sleep type, an arbitrary sleep parameter determined on the basis of the sleep state of the user.

Specifically, the benefit provision unit 1043 of the server 10 updates the record of the user in the user table 1012 and the record of the character that the user owns in the deck table 1013 in accordance with the acquired benefit offer.

### <Sleep Measurement Processing>

The sleep measurement processing is processing of measuring a sleep state of the user using the measurement terminal 40 and storing the sleep state in the storage unit 401 of the measurement terminal 40. In a case where a plurality of measurement terminals 40A, 40B, and 40C are present, the sleep measurement processing is executed in each of the plurality of measurement terminals 40A, 40B, and 40C.

### <Overview of Sleep Measurement Processing>

The sleep measurement processing is a series of processes of measuring a sleep state of the user as sleep information and storing the sleep information in the sleep table 4013 in the measurement terminal 40.

### <Details of Sleep Measurement Processing>

The measurement terminal 40 constantly executes sensing using the sleep sensor 4086 and detects user's going to bed from sensing data (motions of the terminal or brightness or sound in the surroundings, for example).

The sleep measurement processing may be automatically started by detecting the user turning on a power source of the measurement terminal 40.

The measurement terminal 40 may receive an input operation indicating that the user is going to bed and use the input operation as a trigger to start the sleep measurement processing. The input operation indicating that the user is going to bed may be received on the basis of a switch or the like provided in the measurement terminal 40. An input operation based on an operation of interrupting the game or an operation of putting the character to sleep (stroking the character via the touch panel 206, turning off the light that the character is using, or the like) may be received by the user terminal 20, and reception of the input operation via the communication IF may be used as a trigger to start the sleep measurement processing in the measurement terminal 40.

The sleep measurement unit 4042 of the measurement terminal 40 provides an instruction to execute sensing to the sleep sensor 4086. Specifically, the sleep sensor 4086 senses motions of the measurement terminal 40 and biological information such as a heart rate of the user. The sleep measurement unit 4042 of the measurement terminal 40 acquires sensing data from the sleep sensor 4086.

The sleep measurement unit 4042 of the measurement terminal 40 newly stores the sleep information including a sleep pattern indicating a bedtime and a wake-up time in the column of the sleep measurement information of the record in the sleep table 4013 on the basis of the sensing data acquired from the sleep sensor 4086.

Information regarding sleep quality of the user may be included in the sleep information to be stored in the sleep measurement information. At this time, the measurement terminal 40 calculates information regarding sleep quality of the user from the biological information of the user detected by the sleep sensor 4086. The information regarding the sleep of the user may be calculated by employing a known sleep analyzing function of an existing multifunctional device that functions as the measurement terminal 40.

The sleep measurement unit 4042 of the measurement terminal 40 stores the sleep information including the information regarding the sleep quality in the column of the sleep measurement information in the sleep table 4013.

Information regarding the date and time when the sleep information is measured may be included in the sleep information to be stored in the sleep measurement information. Specifically, information regarding a date and time when the sleep measurement processing is executed (a start date and time, for example) may be included in the sleep measurement information.

The sleep measurement unit 4042 of the measurement terminal 40 may be configured to store the sleep information including the sensing data acquired from the sleep sensor 4086 in the column of the sleep measurement information in the sleep table 4013.

The sleep measurement unit 4042 of the measurement terminal 40 defines a period from going to bed to waking up of the user as one measurement period, defines sleep information measured in each measurement period as one record, and stores the record in the sleep table 4013.

### <Sleep Synchronization Processing>

The sleep synchronization processing is processing of synchronizing information regarding sleep of the user stored in the measurement terminal 40 with sleep information of the user stored in the server 10. Hereinafter, details of the sleep synchronization processing will be described using the flowchart in FIG. 13 and examples of display screens in FIGS. 15 and 16.

The sleep synchronization processing is processing of synchronizing information regarding sleep of the user stored in a plurality of measurement terminals 40 with sleep information of the user stored in the server 10.

### <Overview of Sleep Synchronization Processing>

The sleep synchronization processing is a series of processes of acquiring information regarding sleep of the user stored in the measurement terminal 40, acquiring play information of the user stored in the server 10, associating the information regarding the sleep of the user with the play information, determining a play result, and presenting the play result to the user.

### <Details of Sleep Synchronization Processing>

In Step S501, the synchronization unit 1048 of the server 10 starts the sleep synchronization processing.

As for the sleep synchronization processing, the processing may be started by being triggered by the server 10 and the measurement terminal 40 becoming communicable.

As for the sleep synchronization processing, the processing may be started by being triggered by the measurement terminal 40 becoming communicable with the user terminal 20.

As for the sleep synchronization processing, the touch panel 206 of the user terminal 20 to which the measurement terminal 40 is connected may receive a predetermined operation from the user, and the processing may be started by being triggered by the input operation. For example, the sleep synchronization processing may be started by the user pressing a synchronization button displayed on the touch panel 206 of the user terminal 20 when the measurement terminal 40 is connected to the user terminal 20.

In Step S502, the synchronization unit 1048 of the server 10 acquires sleep measurement information corresponding to one time or a plurality of times from the measurement terminal 40. The synchronization unit 1048 of the server 10 transmits a request regarding the acquisition of the sleep measurement information to the measurement terminal 40. When the control unit 404 of the measurement terminal 40 receives the request, the control unit 404 searches a record with a synchronization flag in which information indicating that synchronization has not been performed is stored from the sleep table 4013 and acquires the sleep measurement information. The control unit 404 of the measurement terminal 40 transmits the acquired sleep measurement information as a response to the server 10. The synchronization unit 1048 of the server 10 acquires the sleep measurement information from the measurement terminal 40.

The synchronization unit 1048 of the server 10 may search for the sleep information of the user stored in the user table 1012 and exclude sleep measurement information in a period that has already been stored in the user table 1012 out of the sleep measurement information acquired from the measurement terminal 40. This is because the sleep information is considered to have been acquired by the user terminal 20 or have already been measured by another measurement terminal 40 and the sleep synchronization processing has been performed thereon.

The synchronization unit 1048 of the server 10 may exclude sleep measurement information that is older than a predetermined number of times or a predetermined of number of days out of the sleep measurement information acquired from the measurement terminal 40. In other words, the synchronization unit 1048 of the server 10 may set an upper limit value for the number of times or the number of days for the sleep measurement information acquired from the measurement terminal 40. For example, the synchronization unit 1048 of the server 10 acquires the sleep measurement information with sleep measurement information more than one week ago excluded.

An upper limit value may be set for the number of times or the number of days for the sleep measurement information that the measurement terminal 40 can store. The control unit 404 of the measurement terminal 40 may set an upper limit value of the number of times or the number of days for the sleep measurement information to be transmitted as a response to the server 10. Specifically, the sleep measurement information that is older than a predetermined number of days may not be transmitted to the server 10.

It is thus possible to encourage the user to synchronize the measurement terminals on a regular basis.

The upper limit value of the number of times or the number of days may be changed in accordance with how often the user uses the user terminal 20, how often the user plays the game, or the like. The upper limit value of the number of times or the number of days may be changed in accordance with play information of the user such as a progress state of the played game. In other words, the upper limit value of the number of times or the number of days is defined in accordance with the play information.

Note that in a case where the upper limit of the number of times or the number of days is changed, a notification indicating that the upper limit value of the number of times or the number of days will change may be provided on the display 2062 of the user terminal 20. It is thus possible to encourage the user to synchronize the measurement terminals on a regular basis.

In regard to the sleep measurement information transmitted as a response to the server 10, the control unit 404 of the measurement terminal 40 stores information indicating that the sleep measurement information has been acquired in an item of a synchronization flag of a corresponding record in the sleep table 4013.

Note that the control unit 404 of the measurement terminal 40 may store the information indicating that the sleep measurement information has been acquired in the item of the synchronization flag after information indicating that the sleep measurement information is acquired or the synchronization has been completed is received from the server 10.

In Step S503, the synchronization unit 1048 of the server 10 searches the user table 1012 and acquires one or a plurality of pieces of play information of the user.

In Step S503, the synchronization unit 1048 of the server 10 may specify a range of play information to be acquired on the basis of a period of the sleep information included in the sleep measurement information acquired in Step S502. For example, play information included in a period from a wake-up time or a bedtime of the oldest sleep information to a wake-up time or a bedtime of the latest sleep information out of the acquired sleep information may be acquired. It is thus possible to reduce the amount of information as a target of the processing.

In Step S504, the synchronization unit 1048 of the server 10 associates the sleep information corresponding to one time or a plurality of times acquired from the measurement terminal 40 in Step S502 and the one or plurality of pieces of play information acquired in Step S503 for each predetermined period. Although the present disclosure will be described on the assumption that the predetermined period is one day for simplicity, it is possible to select, as the predetermined period, a predetermined time interval such as every 6 hours or 8 hours, predetermined multiple days such as two days or three days, or another arbitrary period such as one week or one month.

Specifically, the synchronization unit 1048 of the server 10 determines sleep information for each day on the basis of measurement date information indicating a date when the sleep information has been measured from the sleep information acquired from the measurement terminal 40 and associates the sleep information for each day with a history of play information in play date information indicating a date when the game has been played on each day. In other words, the sleep information including the sleep parameter and the play information including the game parameter are associated on the basis of the measurement date information and the play date information.

The synchronization unit 1048 of the server 10 may include the sleep information of the user that has already been stored in the user table 1012 and perform association with the play information. In other words, sleep information that has already been measured by another terminal such as the user terminal 20 and has subjected to the sleep synchronization processing may be included and associated with the play information.

The synchronization unit 1048 of the server 10 may newly generate play information in a case where the sleep information is present while play information on the day (sleep information present day) when the sleep information is present is not present, and associate the newly generated play information with the sleep information. The synchronization unit 1048 of the server 10 may refer to the play information on the previous day (preceding period) of the sleep information present day, generate the play information as the play information on the sleep information present day, and associate it with the sleep information.

In Step S505, the synchronization unit 1048 of the server 10 determines a play result on the basis of the combination of the associated sleep information and play information for each day. In the present disclosure, a case where a benefit is provided will be illustratively described as an example of the play result.

The sleep score calculation unit 1045 of the server 10 executes the sleep score calculation processing on the basis of the sleep information for each day after the association and calculates a sleep score for each day. The sleep score calculation processing is similar to the sleep score calculation processing in Step S103 in the benefit provision processing. Specifically, the sleep score for each day is calculated on the basis of information regarding whether or not the sleep time, the sleep quality, and the sleep pattern included in the sleep information for each day satisfy the reference sleep pattern.

When the sleep score calculation processing is executed, subtraction may be performed for the sleep scores that are older than a predetermined number of days to reduce the influences of the sleep scores on the play result. For example, it is considered to reduce sleep scores occurring four or more days ago to halves. Similarly, addition may be performed for sleep scores that are later than a predetermined number of days to increase the influences of the sleep scores on the play result. It is thus possible to encourage the user to execute the sleep synchronization processing on the regular basis.

Note that in a case where the sleep synchronization processing has not been executed for a predetermined number of days, a notification indicating that the influences on the play result will be reduced may be provided on the display 2062 of the user terminal 20. Specifically, a notification such as “The sleep score of the sleep information occurring four or more days ago will be reduced to half. Do you have any sleep information that has not been reflected?” is provided to the user.

The synchronization unit 1048 of the server 10 determines a play result on the basis of a combination of the sleep score and the play information. In the present disclosure, a case where a character point is used as an example of the play information will be illustratively described.

The total score calculation unit 1046 of the server 10 executes the total score calculation processing on the basis of the sleep score and the character point and calculates the total score. The total score calculation processing is similar to the total score calculation processing in Step S104 in the benefit provision processing. Specifically, the total score for each day is calculated on the basis of the sleep score and the character point for each day.

The benefit provision unit 1043 of the server 10 specifies and determines the benefit offer to be provided for each day on the basis of the total score for each day.

The benefit provision unit 1043 of the server 10 searches a benefit condition in the benefit master 1017 on the basis of the calculated total score and the like.

Specifically, a record of the benefit condition that satisfies a condition such as a calculated total score or the like is specified. Then, the benefit offer stored in the record is specified and acquired. Note that as the benefit condition in the benefit master 1017, an arbitrary condition based on the character point, the sleep score, or the like can be set instead of the total score.

Note that although the configuration in which the benefit offer is specified on the basis of the total score has been employed in the present disclosure, a configuration in which the benefit offer is specified and determined from the character point and the sleep score without calculating the total score may also be employed.

The synchronization unit 1048 of the server 10 may exclude the benefit offers that have already been provided to the user out of the specified benefit offers. Specifically, in a case where the benefit offer on the target day has already been provided through the sleep synchronization processing with another measurement terminal 40, the benefit provision processing with the user terminal 20, or the like before the sleep measurement information is acquired from the measurement terminal 40, it is preferable to exclude the benefit offer in the current sleep synchronization processing.

In other words, the synchronization unit 1048 of the server 10 is configured to specify and determine only the benefit offer to be provided to the user for the first time on the basis of the sleep measurement information acquired from the measurement terminal 40 in the previous sleep synchronization processing out of the specified benefit offers.

The synchronization unit 1048 of the server 10 specifies the benefit offer to be provided to the user for each day and determines it as a play result. The synchronization unit 1048 of the server 10 provides the determined benefit offer to the user. The provision of the benefit offer to the user is similar to Step S106 in the benefit provision processing.

In Step S506, the synchronization unit 1048 of the server 10 presents the determined play result for each day to the user. The synchronization unit 1048 of the server 10 transmits the determined play result to the user terminal 20. The display 2062 of the user terminal 20 displays the received play result and presents the play result to the user. Note that the user terminal 20 may present the play result to the user through light, sound, or the like.

The user terminal 20 presents the play result while selectively switching different presentation modes in accordance with which of the play result for one day and the play results for a plurality of days the received play result includes.

In a case where the received play result includes the play result for one day, the user terminal 20 presents the play result in a first presentation mode.

An example of a screen that presents the play result in the first presentation mode is illustrated in FIG. 15. As the presentation screen, information related to the play result including a benefit offer 701, a sleep time 702, a bedtime and a wake-up time 703, a sleep score 704, a sleep type 705, a date 706, and the like which are information based on the play result determined through the sleep synchronization processing is presented to the user on the game screen 70 of the display 2062 of the user terminal 20. The user can check the play result including the date 706 of the sleep information synchronized through the sleep synchronization processing and the benefit offer 701 through the display 2062 of the user terminal 20.

In a case where play results for a plurality of days are included as the received play results, the user terminal 20 presents the play results in a second presentation mode.

An example of the screen that presents the play results in the second presentation mode is illustrated in FIG. 15. As the presentation screen, information based on the play results determined in the sleep synchronization processing is list-displayed in the form of a list 71 on the game screen 70 of the display 2062 of the user terminal 20. In each item in the list, information regarding the play result for each day including the benefit offer 711, the sleep time 712, the sleep score 714, the date 716, and the like that are information based on the play result is presented as a list to the user. Note that a bedtime and a wake-up time, a sleep type, and the like may be included in each item of the list.

The user can display and check the list of the play results including the date 716 of the sleep information synchronized through the sleep synchronization processing and the benefit offer 711 through the display 2062 of the user terminal 20.

The user terminal 20 may present the play results for the plurality of days in an order based on the sleep scores used to determine the play results as the second presentation mode. Specifically, the play results with better sleep scores may be presented in descending order.

The user terminal 20 may present the play results in the plurality of days in an order based on the total scores used to determine the play results and determined from the play information and the sleep information as the second presentation mode. Specifically, the play results with better total scores may be presented in the descending order.

The user terminal 20 may present the play results for the plurality of days in an order based on dates in a corresponding predetermined period as the second presentation mode. Specifically, the play results may be presented in the order from the later play results.

The user terminal 20 may present the play results for the plurality of days in descending order from the better play results as the second presentation mode.

The user terminal 20 may present the play results for the plurality of days in an order based on awards given to the user in accordance with the play results as the second presentation mode. For example, the presentation may be made in the descending order from the higher rarities of the awards. The awards include a character point.

The user terminal 20 may present a play result corresponding to one or a plurality of days for which sleep information has not been able to be acquired to determine the play results from among the play results for the plurality of days as the second presentation mode. The user can thus check the play result on the day when the play information has been generated.

The user terminal 20 may present play results corresponding to one day or a plurality of days indicating satisfactory sleep states of the user on the basis of the sleep information used to determine the play results from among the play results for the plurality of days as the second presentation mode.

### <Basic Hardware Configuration of Computer>

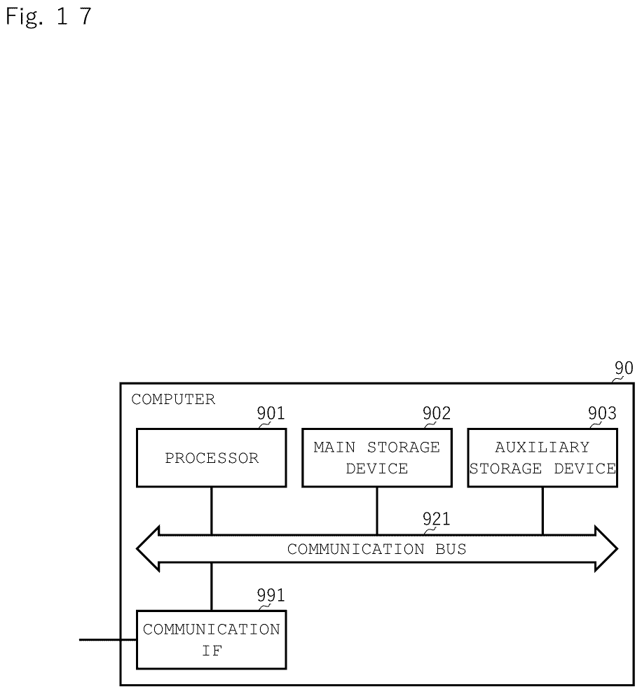

FIG. 17 is a block diagram illustrating a basic hardware configuration of a computer 90. The computer 90 includes at least a processor 901, a main storage device 902, an auxiliary storage device 903, and a communication IF 991 (interface).

These are electrically connected to each other via a communication bus 921.

The processor 901 is a hardware for executing a command set described in a program. The processor 901 is configured of an arithmetic operation device, a register, a peripheral circuit, and the like.

The main storage device 902 is adapted to temporarily store a program and data processed by the program and the like. For example, the main storage device 902 is a volatile memory such as a dynamic random access memory (DRAM).

The auxiliary storage device 903 is a storage device adapted to save data and programs. For example, the auxiliary storage device is a flash memory, a hard disc drive (HDD), a magneto-optical disk, a CD-ROM, a DVD-ROM, a semiconductor memory, or the like.

The communication IF 991 is an interface adapted to input and output signals to communicate with other computers via a network using a wired or wireless communication standard.

The network is configured by various mobile communication systems constructed by the Internet, a LAN, a wireless base station, and the like. For example, the network includes 3G, 4G, and 5G mobile communication systems, Long Term Evolution (LTE), a wireless network (Wi-Fi (registered trademark), for example) that can be connected to the Internet by a predetermined access point, and the like. In a case where connection is established in a wireless manner, communication protocols include, for example, Z-Wave (registered trademark), ZigBee (registered trademark), Bluetooth (registered trademark), and the like. In a case where connection is established in a wired manner, the network includes a network directly connected through a universal serial bus (USB) cable or the like.

Note that it is possible to virtually implement the computer 90 by providing an entirety or a part of the hardware configuration in a plurality of computers 90 and connecting the computers 90 to each other via a network. In this manner, the computer 90 is a concept that includes not only a computer 90 accommodated in a single casing or case but also a virtualized computer system.

### <Basic Functional Configuration of Computer 90>

A functional configuration of the computer implemented by the basic hardware configuration (FIG. 17) of the computer 90 will be described. The computer includes at least functional units of a control unit, a storage unit, and a communication unit.

Note that the functional units included in the computer 90 can also be implemented by an entirety or a part of each functional unit being provided in a plurality of computers 90 connected to each other via a network in a dispersed manner. The computer 90 is a concept that includes not only a single computer 90 but also a virtualized computer system.

The control unit is implemented by the processor 901 reading various programs stored in the auxiliary storage device 903, deploying the programs on the main storage device 902, and executing processing in accordance with the programs. The control unit can implement functional units that perform various kinds of information processing in accordance with the types of the programs. In this manner, the computer is implemented as an information processing device that performs information processing.

The storage unit is implemented by the main storage device 902 and the auxiliary storage device 903. The storage unit stores data, various programs, and various databases.

Also, the processor 901 can secure a storage region corresponding to the storage unit in the main storage device 902 or the auxiliary storage device 903 in accordance with the programs. In addition, the control unit can cause the processor 901 to execute addition, update, and deletion processing of data stored in the storage unit in accordance with the various programs.

The databases means relational databases and are adapted to manage data groups in the chart format structurally defined by rows and columns and called tables or masters in a mutually associated manner. In a database, a chart is called a table or a master, a chart column is called a column, and a chart row is called a record. In a relational database, it is possible to set relationships among the tables or masters and to associate them.

Although a column as a main key for uniquely specifying a record is typically set in each table or each master, the setting of the main key for the column is not essential. The control unit can cause the processor 901 to execute addition, deletion, and update of a record with respect to a specific table or master stored in the storage unit in accordance with the various programs.

Note that the database and the master in the present disclosure may include an arbitrary data structure (a list, a dictionary, an associative array, an object, or the like) in which information is structurally defined. It is assumed that the data structure also includes data that can be regarded as a data structure by combining data with functions, classes, methods, or the like described in an arbitrary programming language.

The communication unit is implemented by the communication IF 991. The communication unit implements a function of communicating with other computers 90 via a network. The communication unit can receive information transmitted from other computers 90 and input the information to the control unit. The control unit can cause the processor 901 to execute information processing on the received information in accordance with the various programs. Also, the communication unit can transmit information output from the control unit to other computers 90.

### APPENDICES

Matters described in each of the above embodiments will be appended below.

### Appendix 1

A game program which a computer that includes a processor and a storage unit and is capable of acquiring a sleep state of a user as sleep information is caused to execute, the game program causing the processor to execute: a sleep acquisition step (S502) of acquiring the sleep information of the user corresponding to a plurality of times; a play acquisition step (S503) of acquiring play information of the user in a game; an association step (S504) of associating the sleep information corresponding to the plurality of times acquired in the sleep acquisition step with the play information acquired in the play acquisition step; and a determination step (S505) of determining a plurality of play results on the basis of a combination of the play information and the sleep information corresponding to the plurality of times associated in the association step.

It is thus possible to reflect the measured sleep information corresponding to the plurality of times in the game. It is possible to continuously encourage the user to make an effort for good sleep habits.

### Appendix 2

The game program according to Appendix 1, in which the sleep acquisition step is a step of acquiring the sleep information including a measurement date information indicating a date when the sleep information was measured and a sleep parameter determined on the basis of a sleep state of the user for the sleep information corresponding to each of the plurality of times, the play acquisition step is a step of acquiring the play information including play date information indicating a date on which the game has been played and a game parameter based on results of the play, the association step is a step of associating the sleep parameter and the game parameter on the basis of the measurement date information acquired in the sleep acquisition step and the play date information acquired in the play acquisition step, and the determination step is a step of determining the plurality of play results on the basis of a combination of the sleep parameter and the game parameter associated in the association step.

It is thus possible to reflect the measured sleep information corresponding to the plurality of times in the game. It is possible to continuously encourage the user to make an effort for good sleep habits.

### Appendix 3

The game program according to Appendix 1 or 2, in which the association step includes, in a period when the play information to be associated is not present, a generation step of generating the play information in the period, and a step of associating the generated play information and the sleep information corresponding to the plurality of times acquired in the sleep acquisition step.

It is thus possible to associate the play information and the sleep information even in a predetermined period when there is no play information. It is possible to encourage the user to play the game without feeling a stress.

### Appendix 4

The game program according to Appendix 3, in which the generation step is a step of generating the play information on the basis of play information in a period preceding the period.

It is thus possible to associate the play information and the sleep information even in a predetermined period when there is no play information. It is possible to encourage the user to play the game without feeling a stress.

### Appendix 5

The game program according to any one of Appendices 1 to 4, in which the sleep acquisition step includes a step of acquiring sleep information of the user corresponding to a plurality of times from a plurality of measurement terminals capable of measuring a sleep state of the user as sleep information.

It is thus possible to reflect the sleep information corresponding to the plurality of times measured by the plurality of measurement terminals in the game. It is possible to continuously encourage the user who uses the plurality of measurement terminals to make an effort for good sleep habits.

### Appendix 6

The game program according to any one of Appendices 1 to 5, in which the determination step includes a step of determining a first play result in a first period on the basis of the play information and the sleep information in the first period, and a step of determining a second play result in a second period on the basis of the play information and the sleep information in the second period, the first period is a period later than the second period, and an influence of the sleep information on the first play result is greater than an influence of the sleep information on the second play result.

It is thus possible to cause later sleep information to have a greater influence on the play result and to encourage the user to perform synchronization of the measurement terminals on a regular basis.

### Appendix 7

The game program according to any one of Appendices 1 to 6, in which the game program causes the processor to execute: a notification step of providing a notification indicating that an influence of the sleep information on the play result becomes small to the user in a case where the sleep acquisition step has not been executed for a predetermined period of time or more.

It is thus possible to encourage the user to perform synchronization of the measurement terminals on a regular basis.

### Appendix 8

The game program according to any one of Appendices 1 to 7, in which the sleep acquisition step is a step of acquiring the sleep information corresponding to a predetermined number of times of the user, and the predetermined number of times is a value defined in accordance with progress of a game.

It is thus possible to encourage the user to play the game.

### Appendix 9

The game program according to any one of Appendices 1 to 8, in which the game program causes the processor to execute: a presenting step (S506) of presenting the play result determined in the determination step to the user, in which the presenting step is a step of performing presentation by selectively switching a first presenting step of presenting one play result in a first presentation mode to the user in a case where a play result determined in the determination step is the one play result, and a second presenting step of presenting a plurality of play results in a second presentation mode to the user in a case where play results determined in the determination step are the plurality of play results.

It is thus possible to continuously encourage the user to make an effort for good sleep habits while allowing the user to enjoy the presented content.

### Appendix 10

The game program according to Appendix 9, in which the second presentation mode in the second presenting step is at least one of presentation modes including a presentation mode in which the plurality of play results are presented in an order based on sleep scores determined by the sleep information used to determine the play results, a presentation mode in which the plurality of play results are presented in an order based on a total score determined by the play information and the sleep information used to determine the play results, a presentation mode in which the plurality of play results are presented in an order based on dates, a presentation mode in which the plurality of play results are presented in descending order from a satisfactory one of the play results, a presentation mode in which the plurality of play results are presented in an order based on awards given to the user depending on the play results, a presentation mode in which one or more of the play results, for which the sleep information has not been able to be acquired in determination of the play results, from among the plurality of play results are presented, and a presentation mode in which one or more of the play results indicating satisfactory sleep states of the user are presented on the basis of sleep information used to determine the play results from among the plurality of play results.

It is thus possible to continuously encourage the user to make an effort for good sleep habits while allowing the user to enjoy the presented content.

### Appendix 11

The game program according to any one of Appendices 1 to 10, in which the sleep acquisition step is a step of acquiring the sleep information corresponding to the plurality of times from a measurement terminal.

It is thus possible to reflect the sleep information corresponding to one time or a plurality of times measured by the measurement terminal in the game. It is possible to continuously encourage a user who uses the measurement terminal to make an effort for good sleep habits.

### Appendix 12

The game program according to Appendix 11, in which the game program causes the processor to execute: a step (S502) of storing information indicating that the sleep information has been acquired in an associated manner with the sleep information corresponding to the plurality of times in the measurement terminal in accordance with the sleep information corresponding to the plurality of times acquired in the sleep acquisition step, and the sleep acquisition step is a step of acquiring the sleep information corresponding to the plurality of times that is not associated with the information indicating that the sleep information has been acquired from the measurement terminal.

It is thus possible to reflect the sleep information except for the sleep information that has already been acquired in the game. It is possible to prevent the sleep information that has already been reflected from being reflected in the game.

### Appendix 13

The program according to any one of Appendices 1 to 10, in which the sleep acquisition step is a step of acquiring the sleep information corresponding to the plurality of times from a program that is different from the game program.

It is possible to reflect the sleep information corresponding to the plurality of times acquired by the different game program in the game. It is possible to continuously encourage the user to make an effort for good sleep habits.

### Appendix 14

An information processing device including: a processor; and a storage unit, in which the processor is caused to execute a sleep acquisition step (S502) of acquiring the sleep information of the user corresponding to a plurality of times, a play acquisition step (S503) of acquiring play information of the user in a game, an association step (S504) of associating the sleep information corresponding to the plurality of times acquired in the sleep acquisition step with the play information acquired in the play acquisition step, and a determination step (S505) of determining a plurality of play results on the basis of a combination of the play information and the sleep information corresponding to the plurality of times associated in the association step.

It is thus possible to reflect the measured sleep information corresponding to one time or a plurality of times in the game. It is possible to continuously encourage the user to make an effort for good sleep habits.

### Appendix 15

An information processing method that is executed by a computer including a processor and a storage unit, the method including causing the processor to execute: a sleep acquisition step (S502) of acquiring the sleep information of the user corresponding to a plurality of times; a play acquisition step (S503) of acquiring play information of the user in a game; an association step (S504) of associating the sleep information corresponding to the plurality of times acquired in the sleep acquisition step with the play information acquired in the play acquisition step; and a determination step (S505) of determining a plurality of play results on the basis of a combination of the play information and the sleep information corresponding to the plurality of times associated in the association step.

It is thus possible to reflect the measured sleep information corresponding to one time or a plurality of times in the game. It is possible to continuously encourage the user to make an effort for good sleep habits.

### Appendix 16

An information processing system including: an information processing device that includes a processor and a storage unit; and a measurement terminal, in which the information processing system executes: a sleep acquisition step (S502) of acquiring the sleep information of the user corresponding to a plurality of times from the measurement terminal; a play acquisition step (S503) of acquiring play information of the user in a game; an association step (S504) of associating the sleep information corresponding to the plurality of times acquired in the sleep acquisition step with the play information acquired in the play acquisition step; and a determination step (S505) of determining a plurality of play results on the basis of a combination of the play information and the sleep information corresponding to the plurality of times associated in the association step.

It is thus possible to reflect the sleep information corresponding to one time or a plurality of times measured by the measurement terminal in the game. It is possible to continuously encourage a user who uses the measurement terminal to make an effort for good sleep habits.

1. A non-transitory computer-readable storage medium storing computer-readable instructions thereon which, when executed by a computer that includes a processor and a memory and is capable of acquiring a sleep state of a user as sleep information, causes the computer to perform a method, the method comprising:
acquiring the sleep information of the user a plurality of times;
acquiring play information of the user in a game;
associating the sleep information with the play information; and
determining a plurality of play results based on each combination of the associated play information and sleep information.

2. The non-transitory computer-readable storage medium according to claim 1, further comprising:
acquiring the sleep information including measurement date information indicating a date when the sleep information was measured and a sleep parameter determined on the basis of a sleep state of the user for the sleep information corresponding to each of the plurality of times;
acquiring the play information including play date information indicating a date on which the game was played and a game parameter based on results of the play;
associating the sleep parameter and the game parameter on the basis of the acquired measurement date information and the acquired play date information; and
determining the plurality of play results on the basis of a combination of the associated sleep parameter and game parameter.

3. The non-transitory computer-readable storage medium according to claim 1, further comprising:
in a period that the play information to be associated is not present,
generating the play information in the period, and
associating the generated play information and the sleep information.

4. The non-transitory computer-readable storage medium according to claim 3, further comprising:
generating the play information on the basis of play information in another period preceding the period.

5. The non-transitory computer-readable storage medium according to claim 1, further comprising:
acquiring the sleep information of the user the plurality of times from a plurality of measurement terminals capable of measuring a sleep state of the user as sleep information.

6. The non-transitory computer-readable storage medium according to claim 1, further comprising:
determining a first play result in a first period on the basis of the play information and the sleep information in the first period, and
determining a second play result in a second period on the basis of the play information and the sleep information in the second period,
the first period is a period later than the second period, and
an influence of the sleep information on the first play result is greater than an influence of the sleep information on the second play result.

7. The non-transitory computer-readable storage medium according to claim 1, further comprising:
providing a notification indicating that an influence of the sleep information on the play result becomes less influential to the user in a case where acquiring the sleep information has not been executed for a predetermined period of time or more.

8. The non-transitory computer-readable storage medium according to claim 1, further comprising:
acquiring the sleep information of the user a predetermined number of times,
wherein the predetermined number of times is a value defined in accordance with progress of a game.

9. The non-transitory computer-readable storage medium according to claim 1, further comprising:
presenting the play result to the user,
wherein presenting the play result includes performing presentation by selectively switching
presenting one play result in a first presentation mode to the user in a case where a play result is the one play result, and
presenting a plurality of play results in a second presentation mode to the user in a case where play results are the plurality of play results.

10. The non-transitory computer-readable storage medium according to claim 9, wherein the second presentation mode includes at least one of
a presentation mode in which the plurality of play results are presented in an order based on sleep scores determined by the sleep information used to determine the play results,
a presentation mode in which the plurality of play results are presented in an order based on a total score determined by the play information and the sleep information used to determine the play results,
a presentation mode in which the plurality of play results are presented in an order based on dates,
a presentation mode in which the plurality of play results are presented in descending order from a satisfactory one of the play results,
a presentation mode in which the plurality of play results are presented in an order based on awards given to the user depending on the play results,
a presentation mode in which one or more of the play results, for which the sleep information has not been able to be acquired in determination of the play results, from among the plurality of play results are presented, and
a presentation mode in which one or more of the play results indicating satisfactory sleep states of the user are presented on the basis of sleep information used to determine the play results from among the plurality of play results.

11. The non-transitory computer-readable storage medium according to claim 1, further comprising:
acquiring the sleep information corresponding to the plurality of times from a measurement terminal.

12. The non-transitory computer-readable storage medium according to claim 11, further comprising:
storing information indicating that the sleep information has been acquired in an associated manner with the sleep information corresponding to the plurality of times in the measurement terminal in accordance with the sleep information acquired the plurality of times, and
acquiring the sleep information corresponding to the plurality of times that is not associated with the information indicating that the sleep information has been acquired from the measurement terminal.

13. The non-transitory computer-readable storage medium according to claim 1, further comprising:
acquiring the sleep information the plurality of times from a program that is different from the non-transitory computer-readable storage medium.

14. An information processing device comprising:
a processor; and
a memory,
wherein the processor in combination with the memory are configured to execute the following:
acquiring sleep information of a user a plurality of times;
acquiring play information of the user in a game;
associating the sleep information with the play information, and
determining a plurality of play results based on each combination of the associated play information and sleep information.

15. An information processing method that is executed by a computer including a processor and a memory, the method comprising:
acquiring sleep information of a user a plurality of times;
acquiring play information of the user in a game;
associating the sleep information with the play information; and
determining a plurality of play results based on each combination of the associated play information and sleep information.

16. An information processing system, comprising:
a measurement terminal; and
an information processing device that includes a processor and a memory,
wherein the processor in combination with the memory are configured to:
acquire the sleep information of a user a plurality of times from the measurement terminal,
acquire play information of the user in a game,
associating the sleep information with the play information, and
determining a plurality of play results based on each combination of the play information and the sleep information.

17. The method of claim 15, further comprising:
acquiring the sleep information including measurement date information indicating a date when the sleep information was measured and a sleep parameter determined on the basis of a sleep state of the user for the sleep information corresponding to each of the plurality of times;
acquiring the play information including play date information indicating a date on which the game was played and a game parameter based on results of the play;
associating the sleep parameter and the game parameter on the basis of the acquired measurement date information and the acquired play date information; and
determining the plurality of play results on a basis of each combination of the associated sleep parameter and game parameter.

18. The method of claim 15, further comprising:
in a period that the play information to be associated is not present,
generating the play information in the period, and
associating the generated play information and the sleep information.

19. The method of claim 18, further comprising:
generating the play information on the basis of play information in another period preceding the period.

20. The method of claim 15, further comprising:
acquiring the sleep information of the user the plurality of times from a plurality of measurement terminals capable of measuring a sleep state of the user as sleep information.
